(19) United States
(12) Patent Application Publication
(10) Pub. No.: US 2026/0245732 A1
(43) Pub. Date: Aug. 20, 2026

(54) A COMPUTER IMPLEMENTED METHOD AND A DEVICE FOR DETERMINING RISK OF A SUBJECT DEVELOPING OVERT HEPATIC ENCEPHALOPATHY OVER TIME AND A COMPUTER IMPLEMENTED METHOD OF TRAINING A MATHEMATICAL MODEL

(71) Applicants: UCL BUSINESS LTD, London (GB); INCLIVA BIOMEDICAL RESEARCH INSTITUTE, Valencia (ES)

(72) Inventors: Rajiv JALAN, London (GB); Maria Pilar BALLESTER, Valencia (ES); Juan Antonio CARBONELL, Valencia (ES)

(73) Assignees: UCL BUSINESS LTD, London (GB); INCLIVA BIOMEDICAL RESEARCH INSTITUTE, Valencia (ES)

(21) Appl. No.: 19/164,814

(22) PCT Filed: Mar. 12, 2024
(86) PCT No.: PCT/GB2024/050668

§ 371 Date: Sep. 12, 2025

(30) Foreign Application Priority Data
Mar. 13, 2023 (GB) 2303652.8

(51) Int. Cl. G16H 50/30; G06N 7/01; G06N 20/00; G16H 10/40; G16H 10/60; G16H 50/20

(52) U.S. Cl. CPC G16H 50/30; G06N 7/01; G06N 20/00; G16H 10/40; G16H 10/60; G16H 50/20

(57) ABSTRACT

There is provided a computer implemented method of determining risk of a subject developing overt hepatic encephalopathy, OHE, over time. The method includes accepting input of values for blood ammonia level and blood albumin level and optionally the gender of the subject, diabetes status of the subject, and blood creatinine level for the subject into a mathematical model. The method further comprises the mathematical model assessing and outputting the risk of the subject developing OHE over time.

### FIELD OF THE INVENTION

The present invention relates to a computer implemented method of determining risk of a subject developing overt hepatic encephalopathy, OHE, over time. The present invention further relates to a device for determining risk of a subject developing OHE over time and to a training method to determine risk of a subject developing OHE over time. The invention may be used to select patients for therapy to prevent the occurrence of OHE and/or as a surrogate marker to assess response to therapy.

### BACKGROUND OF THE INVENTION

When patients with cirrhosis develop an episode (or first episode) of OHE, their health-related quality of life declines and the risk of hospitalization and death is abruptly increased [1-2]. Minimal hepatic encephalopathy (mHE) has been described as a condition that predisposes to OHE and can be present in up to 80% of patients with stable cirrhosis [3-4].

The diagnosis of mHE is currently based on abnormalities in neurophysiological, neuropsychological or psychophysical tests, which evaluate different elements of brain function. Patient performance in these tests has been suggested to predict the risk of future OHE and its associated mortality [5]. Accordingly, the American and the European Association for the Study of the Liver (AASLD and EASL) recommend that patients with cirrhosis should be evaluated for the presence of mHE using one of these tests [6-7]. However, data regarding their sensitivity and specificity, as well as prognostic and pathophysiological correlates are limited because of lack of widespread availability, required clinical expertise, and the ability of patients to perform these tests due to other comorbidities, sickness, and debility [8-9].

Ammonia has long been recognised as a gut-derived neurotoxin that plays an important role in the pathogenesis of HE [10]. It is involved in several pathophysiological processes such as astrocytic swelling, astrocytic senescence, neuronal cell death, neuroinflammation, mitochondrial dysfunction and altered cerebral bioenergetics leading to impaired glioneuronal communication, which results in cognitive dysfunction [11-12]. In the setting of outpatients with clinically stable cirrhosis, it has recently been demonstrated that hyperammonaemia is associated with increased risk of hospitalization with liver-related complications and mortality. An ammonia level >1.4 times the upper limit of normal for the reference laboratory determine the high-risk group [13]. Accordingly, emerging evidence supports the utility of ammonia for risk stratification. However, the role of ammonia measurement in outpatients with cirrhosis is not clear cut and its utility in predicting development of OHE has not been evaluated [6-7]. Moreover, some patients, such as children, can tolerate an abnormally high level of blood ammonia and this can make ammonia unreliable when used as a predictor.

At the World Congress of Gastroenterology in 1998, the Hepatic Encephalopathy Consensus Group proposed the Psychometric Hepatic Encephalopathy Score, PHES, test as the gold standard for diagnosing mHE and as a guideline for clinical trials in HE [14]. Since then, PHES and many other neuropsychological, neurophysiological and psychophysical tools have been developed for the diagnosis of mHE [15] and hence the risk of OHE. Additionally, Critical Flicker Frequency, CFF, is defined as the frequency at which flickering light can be perceived as continuous and it is used to assess the processing of temporal vision. This can also be used to predict the risk of OHE.

The Child-Pugh-Turcotte (CP) score and the model for end-stage liver disease (MELD) score are the most utilised non-invasive tools for prediction of survival in cirrhotic patients but are limited by interobserver subjectivity and their initial derivations in predicting survival after surgery and transjugular intrahepatic portosystemic shunt (TIPS), respectively [16-17]. Furthermore, the composite features of the MELD score (bilirubin, albumin, international normalised ratio (INR) and creatinine) reflect incomplete facets of the pathophysiology of cirrhotic portal hypertension that are restricted to liver synthetic dysfunction and renal insufficiency and therefore, their utility for the prediction of OHE is limited.

It is desirable to provide an alternative test that can give an indication of whether OHE is likely to be developed by a patient.

### SUMMARY OF THE INVENTION

The invention is defined in the independent claims, to which reference should now be made. Further features are set out in the dependent claims.

According to an aspect of the invention, there is provided a computer implemented method of determining risk of a subject developing overt hepatic encephalopathy, OHE, over time. The method comprises accepting input of values for blood ammonia level and blood albumin level and optionally the gender of the subject, diabetes status of the subject, and blood creatinine level for the subject into a mathematical model. The method further comprises the mathematical model assessing and outputting the risk of the subject developing OHE over time.

The subject may be a patient in a hospital, an outpatient with cirrhosis, for example liver cirrhosis and/or clinically stable cirrhosis or any other suitable person. The subject may be diagnosed with mHE or other comorbidities.

The mathematic model assessing and outputting the risk of the subject developing OHE over time may include any suitable time frame. For example, the risk of developing OHE within 1 year may be assessed. Of course, any time frame may be used. The probability of the subject developing OHE within one, two, three, four and/or 5 years may be assessed. Additionally or alternatively, the model may output a median survival time for the subject. A suitable time frame may be, for example, a time frame in which therapeutic drugs may be administered to a patient to prevent and/or reduce the symptoms of OHE.

The method may further comprise a step of outputting the risk as a survival function, for example using a graph of probability of developing OHE against time, for instance in a Kaplan Meier curve and/or a table of probability of developing OHE against time. The graph may display on one axis a probability that the subject develops OHE and on another axis a time frame, in for example days. The graph may show a functional plot, that is a graph of a function, of the probability of developing OHE against time.

The risk may be output as a table containing the probability that the subject develops OHE in a time frame. For example, the table may output the probability of the subject developing OHE in one, two, three, four and/or 5 years. Additionally or alternatively, the model may output a median survival time for the subject.

The model may be any suitable model, for example a statistical model or a machine learning, ML, model such as a deep learning model or Random Survival Forest, RSF. Such a model may preferably be developed using sampling, such as bootstrapping and/or bagging techniques. The machine model may consist of 500 trees, or close to 500 trees. The trees may be decision tress, for example binary decision trees. The machine model may be developed using sampling with replacement, for example, by using a bootstrapping technique. Additionally or alternatively, a bagging technique may be used, for example by bootstrap aggregating.

The ML model assessing the probability of the subject developing OH E over time may use an ensemble survival function generated from subject terminal nodes of trees in the RSF which are associated with the subject, the subject terminal nodes corresponding to the subject inputs.

For example, the input of the subject may be fed into each of the tress in the RSF. Each tree of the RSF may be formed of a series of nodes and branches. The input variables of the subject may be evaluated at the nodes of each tree and follow the branches of the tree until the subject reaches a terminal node. Each terminal node of each tree may have an associated survival function. The subject may be assigned the survival function of the terminal node of each tree used to evaluate the subject variables.

A log-rank statistic may be used as the splitting rule at each node of each tree. Of course, other splitting rules may be used.

The number of variables at each tree may be calculated by taking the square root of the number of input, or predictor, variables and rounding to the closest integer number. For instance, if 5 input variables are provided, the model may evaluate 2 variables at each split or node.

An ensemble survival function may be generated from the subject terminal nodes of the trees. For example, the ensemble survival function may be calculated by averaging the survival function of each terminal node of each tree in the subject is associated with.

The blood ammonia level may be input as a ratio of a blood ammonia level of the subject to an upper limit of normal blood ammonia level, AMM-ULN. The method may include converting an input ammonia level into an AMM-ULN ratio by dividing the input level by a reference laboratory upper limit of normal for ammonia. This allows for variation in testing methods and apparatus and can have a significant effect on improving harmonisation between locations. The AMM-ULN may be calculated from [(AMM-ULN)=plasma ammonia (μmol/L)/upper limit of normal for ammonia (μmol/L). The upper limit of normal for ammonia may be set with reference to a specific laboratory or hospital or testing centre.

The subject or patient or outpatient may have cirrhosis and a previous episode of OHE and the method may assess the risk of recurrent OHE or the subject may have compensated cirrhosis and the model may predict an episode (or first episode) of OHE.

The mathematical model may produce a score, for example based on the probability of developing OHE within the first year of testing. For instance, the score may be the probability that the subject develops OHE within one year. The probability may be the cumulative probability of the subject developing OHE within one year. Of course, any other time frame may be used. For example, the time frame may relate to a time frame in which treatment is required to prevent to the onset of OHE or for treatment of OHE.

The method may further comprise use of the mathematical model to identify patients requiring specific therapy to prevent the occurrence of OHE. Preferably the method compares the risk of the subject developing OHE over time with a threshold and outputs a recommendation or risk categorisation. For example, the model may compare the probability of the subject developing OHE within one year with a threshold. The threshold may be a value associated with the same time frame as the model. For example, if the model outputs the risk or probability of the subject developing OH E within one year, the threshold value will be a suitable value for assessing risk within one year.

The threshold value may be in the form of a probability, for instance in a range between 0 and 1 and/or as a percentage. The threshold value may be set to 0.7. That is the probability that the subject develops OHE within one year of testing may be 0.7 or 70%. The threshold may define a boundary between recommending the subject for treatment and/or therapy. For example, if the subject has a risk on or above 0.7 the subject may be recommended for treatment and/or therapy. Additionally or alternatively, the subject may be categorised as at risk to developing OHE. The subject may be recommended and/or prescribed drugs based on the risk determination.

Of course, the model may group subjects into categories based on threshold values. For example, a low risk threshold value be 0.1 and any patient with a risk of developing OHE on or below 0.1 may be deemed low risk. A medium risk threshold may be 0.2 and any subject between 0.1 and 0.2 may be deemed medium risk. Any subject with a risk above 0.2 may be deemed high risk. The model may recommend different treatments and/or therapy based on the risk category a subject has been placed in.

The method may further comprise use of the mathematical model as a surrogate marker of response to and/or failure of treatment, preferably by carrying out the method for a subject at predetermined time intervals and comparing the risk of the subject developing OHE overtime. For example, the method may be repeated one or more times within a time frame and the output compared. For instance, the method may be repeated twice within a one or two or three week interval and/or within a one or two or three month interval. The risk of developing OHE within a time frame may be compared between the repeat measurements. The model may act as a surrogate marker if, for example, there is a decrease in risk between repeat measurements. A decrease in risk may indicate the effectiveness of a treatment and/or therapy. Of course, the opposite may also be true. If the risk increases between multiple measurements the model may indicate the failure of a treatment and/or therapy.

Assessment of each subject using the machine model may be done in repeated intervals. For example, in stable outpatients the repeated interval may be every 6 months. In acutely decompensated patients, it may be used every day during admission. As mentioned above, the model may risk stratify patients into low, medium and high-risk groups. Patients with low risk may not need treatment, medium risk patients may require treatment on a case-by-case basis and it may be determined that high risk patients require, for example immediate, treatment.

The input for the method may use a web-based interface or a desktop or a portable test device application. For example, the method may be run on a cloud-based server such as an open source Shiny server. The input may be displayed in the form of a table which accepts values for specified predictor variables. For example, the table may accept input of values for blood ammonia level and blood albumin level and optionally the gender of the subject, diabetes status of the subject, and blood creatinine level for the subject. The values may be input on a local device, such as a medical device or computer, which is connected to a web server. The web-server may execute the method and send the results to the local device. The results may be viewed on a web-based interface such as website or locally offline on the device.

The input variables for the subject may be accessed or retrieved from patient records, for example electronic patient records, EPRs. The subject's input variables may be automatically fed into the model or input by a user. For example, a clinician or technician may input the variables into the model.

According to another aspect of the invention, there is provided a portable test device to determine risk of a subject developing overt hepatic encephalopathy, OHE, over time. The device comprises a blood test sampler and value determiner to take a subject's blood and determine values for blood ammonia level, blood albumin level, and optionally blood creatinine level for the subject. The portable test device may further optionally comprise an input part such as a touch screen or keyboard for inputting the gender of the subject and a diabetes status of the subject if required. A processor of the device or a link to a processor accepts the values for blood ammonia level and blood albumin level and the optional values for gender of the subject, diabetes status of the subject and blood creatinine level and processes them in a mathematical model which assesses the probability of the subject developing OHE based on the time to OHE. The device further comprises an output part such as a screen or network interface, to output the risk.

The blood test sampler may be a strip or disc analyser and/or a blood container with a value determiner configured to detect a specified variable. For example, a detector may be configured to detect ammonia and/or albumin and/or creatinine levels.

The value determiner takes or accepts a subject's blood, from the sampler, and determines values for blood ammonia level, blood albumin level, and optionally blood creatinine level for the subject. The value determiner may function using chemical detection, immunoassay, bioassay and/or spectrophotometry.

The output part of the portable device may output the risk in any way, for instance as a survival function, for example as a displayed graph, for instance in a Kaplan Meier curve, and/or a table of probability of OHE against time. The graph may display on one axis a probability that the subject develops OHE and on another axis a time frame, in for example days. The graph may show a functional plot, that is a graph of a function, of the probability of developing OHE against time.

The risk may be output as a table containing the probability that the subject develops OHE in a time frame. For example, the table may output the probability of the subject developing OHE in one, two, three, four and/or 5 years. The probability may be in a value between 0 and 1 and/or in a percentage. Additionally, or alternatively, the model may output a median survival time for the subject.

The mathematical model used to process the inputs may be substantially similar to, or the same, as the mathematical model described above. For example, the mathematical method may use a machine learning model such as a Random Survival Forest, RSF.

A further aspect of the invention comprises a computer implemented method of training a mathematical model to determine risk of a subject developing overt hepatic encephalopathy, OHE, over time. The method includes inputting, for each of a cohort of subjects, values for blood ammonia level and blood albumin level and optionally the gender of the subject, diabetes status of the subject and, and blood creatinine level for the subject into training software to train a mathematical Model. The method further comprises inputting, for each of the cohort of subjects, an indication if the subject did or did not develop OHE, and an associated time and creating the mathematical model using the input values.

The mathematical model may be a Machine Learning, ML, model such as a Random Survival Forest, RSF.

The Inventors surprisingly found that using all of the variables: Sex, Diabetes Miletus status, Creatinine (mg/dL), AMM-ULN and Albumin (g/dL) to train a mathematic model gave the best model for predicting the development of OHE compared to other standard models known in the prior art.

The training method may further comprise sampling the subject data and growing a plurality of trees, for example, decision trees, one per sample, to create the model.

The training method may further comprise generating a survival function of the probability of developing OHE against time at each terminal node of each decision tree.

The method may further comprise using the model to select patients for therapy to prevent an episode of OHE and/or as a surrogate marker of response, as mentioned above.

A further aspect of the invention comprises a computer program, comprising instructions which when the program is executed on a portable test device or processing device (such as a standalone computer, or a “dumb” PC with a link to a server for example), cause the portable test device or processing device to carry out any of the preceding method definitions or any combination thereof. The computer program may be stored on a computer-readable medium. The computer-readable medium may be non-transitory.

A further aspect of the invention includes a computer program which, when executed by a companion device, causes the companion device to execute a method of an embodiment, for example any of the above methods. The computer program may be stored on a computer-readable medium. The computer-readable medium may be non-transitory.

The invention may be implemented in digital electronic circuitry, or in computer hardware, firmware, software, or in combinations thereof. The invention may be implemented as a computer program or a computer program product, i.e. a computer program tangibly embodied in a non-transitory information carrier, e.g. in a machine-readable storage device or in a propagated signal, for execution by, or to control the operation of, one or more hardware modules.

A computer program may be in the form of a stand-alone program, a computer program portion, or more than one computer program, and may be written in any form of programming language, including compiled or interpreted languages, and it may be deployed in any form, including as a stand-alone program or as a module, component, subroutine, or other unit suitable for use in a data processing environment.

The invention is described in terms of particular aspects. Other embodiments are within the scope of the following claims. For example, the steps of the invention may be performed in a different order and still achieve desirable results. Multiple test script versions may be edited and invoked as a unit without using object-oriented programming technology; for example, the elements of a script object may be organized in a structured database or a file system, and the operations described as being performed by the script object may be performed by a test control program.

The skilled person will appreciate that except where mutually exclusive, a feature described in relation to any one of the above aspects may be applied mutatis mutandis to any other aspect. To be specific, any feature describing the model in the training phase (in which the mathematical model is developed) can of course also apply to the implementation phase (in which risk of an individual subject developing OHE over time is assessed) and/or can apply to the model used in the portable device, and vice versa. Furthermore, except where mutually exclusive, any feature described herein may be applied to any aspect and/or combined with any other feature described herein.

A computer-implemented method according to preferred aspects of the present invention may comprise any combination of the apparatus or computer program aspects. Methods or computer programs according to further aspects may be described as computer-implemented in that they require processing and memory capability.

The apparatus according to preferred aspects is described as configured or arranged to, or simply “to” carry out certain functions. This configuration or arrangement could be by use of hardware or middleware or any other suitable system. In preferred aspects, the configuration or arrangement is by software.

Thus according to one aspect there is provided a program which, when loaded onto at least one computer configures the computer to become the apparatus according to any of the preceding apparatus and/or device definitions or any combination thereof.

According to a further aspect there is provided a computer program or computer program product (or non-transitory storage medium) comprising instructions which when the program is executed on at least one computer causes the at least one computer to carry out the method (steps) according to any of the preceding method definitions or any combination thereof.

In general the computer may comprise the elements listed as being configured or arranged to provide the functions defined. For example this computer may include memory, processing, and a network interface.

A computer program may be deployed to be executed on one module or on multiple modules at one site or distributed across multiple sites and interconnected by a communication network.

Method steps of the invention may be performed by one or more programmable processors executing a computer program to perform functions of the invention by operating on input data and generating output. Apparatus of the invention may be implemented as programmed hardware or as special purpose logic circuitry, including e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions coupled to one or more memory devices for storing instructions and data.

Elements of the invention have been described using terms such as “value determiner” etc. The skilled person will appreciate that functional terms and their equivalents expressed in terms of units may refer to parts of the system that are spatially separate but combine to serve the function defined. Equally, the same physical parts of the system may provide two or more of the functions defined.

For example, separately defined means may be implemented using the same memory and/or processor as appropriate.

### BRIEF DESCRIPTION OF THE DRAWINGS

Reference is made, by way of example only, to the accompanying drawings in which:

FIG. 1A is a flow chart of steps of a method for assessing the risk of a subject developing over hepatic encephalopathy using machine learning;

FIG. 1B is a plot of number of trees in a random forest model against error rate;

FIG. 2A is a graph of Brier score against time used to evaluate the machine model performance using bootstrap cross-validation to predict development of OHE in a validation cohort;

FIG. 2B is another graph Brier score against time used to evaluate the machine model performance using bootstrap cross-validation to predict development of OHE in another validation cohort;

FIG. 3A is a graph of Brier score against time to evaluate the machine learning model performance using bootstrap cross-validation to predict development of OHE. Random forest modelling with input variables was used to compare several models from a training set including PHES, CFF, AMM-ULN, CP, MELD score and an AMMON-OHE model;

FIG. 3B is a plot of variable importance (variable importance measures VIMP) for each of the five variables identified for the machine learning model;

FIG. 4 shows a flow chart of the study population;

FIGS. 5A and 5B show cumulative incidence and Kaplan Meier plots of hospitalization with OHE and mortality in the 4 groups according to PHES and AMM-ULN, respectively;

FIGS. 6A and 6B show cumulative incidence and Kaplan Meier plots of hospitalization with OHE and mortality in the 4 groups according to CFF and AMM-ULN, respectively;

FIG. 7 shows Kaplan Meier plots of survival in patients according to development of OHE. The Kaplan Meier plots demonstrate cumulative probability of overall survival during follow-up;

FIG. 8A shows a graph for prediction for the risk of future OHE using the AMMON-OHE random forest model. The figure shows the probability of OHE in 3 hypothetical patients with high (woman with diabetes mellitus, creatinine of 3.6 mg/Dl, albumin of 2.7 g/Dl and AMM-ULN of 2), medium (man without diabetes, Cr of 1.5 mg/Dl, albumin of 3.2 g/Dl and AMM-ULN of 1.1) and low (man without diabetes, creatinine of 0.7 mg/Dl, albumin of 4.5 g/Dl and AMM-ULN of 0.5) risk;

FIG. 8B is a graph of probability of developing OHE according to the machine learning model prediction at baseline and at 3-6 months, against time;

FIG. 9 shows an example of display on an interface which uses the machine learning model to predict a survival curve (time to develop OHE);

FIG. 10 is a block diagram of a computing device which may be used to implement the machine learning model; and

FIG. 11 is a block diagram of a portable testing device which may be used to implement the machine learning model.

### DETAILED DESCRIPTION

The inventors investigated the role of neuropsychological, psychophysical tests and ammonia for determining the risk of OHE. The inventors surprisingly found that the neuropsychometric tests (current gold standard) were not independent predictors of OHE. Subsequently, the inventors developed a mathematical model, in this case a ML model referred to herein as the AMMON-OHE model, for risk stratification regarding subsequent OHE development. The model may be used, for example, for outpatients with liver cirrhosis. The model may take as an input ammonia and albumin levels of a subject and output a risk to developing OHE over time. The machine learning model may be a fast unified random forest model used to predict future development of OHE.

### Implementation Phase

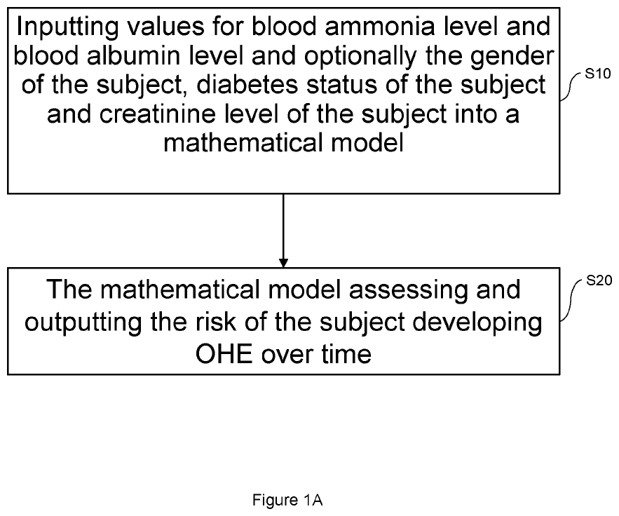

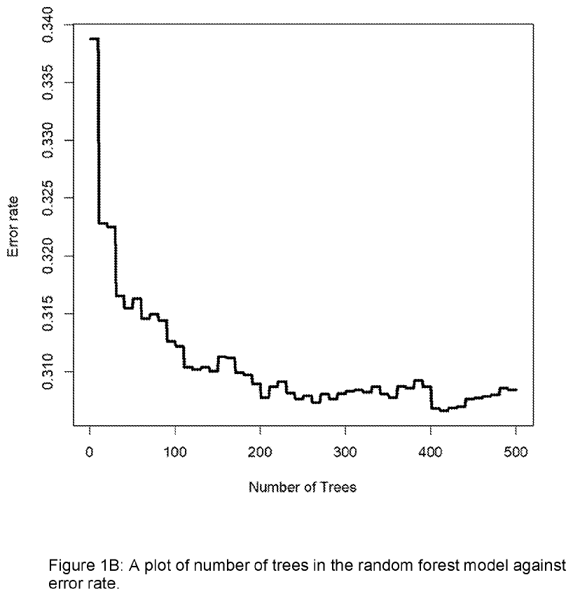

FIG. 1A is a flow chart showing steps of a computer implemented method for determining risk of a subject developing overt hepatic encephalopathy, HE, over time. In step S10, values for blood ammonia levels and blood albumin levels are input into a mathematical model. Optionally, the predictor variables of gender of the subject, diabetes status of the subject and values for blood ammonia level, albumin level, and creatinine level are also input. The mathematical model may be a machine learning, ML model such as a Random Survival Forest. The ML model may be implemented in R project, in one example the inventors used version 4.0.2, R Core, 2021, using the rfsrc function from the randomForestSRC package (Ishwaran H. and Kogalur U. B. 2007 “randomForestSRC”. R statistical software is distributed under the terms of the GNU General Public License, either Version 2, June 1991 or Version 3, June 2007).

The gender of the subject may be input as either Male or Female and the diabetes status as True or False. Albumin levels may be input in units of grams per decilitre (g/dL). The serum (or blood) albumin levels may be obtained from, for example, a serum albumin test or a comprehensive metabolic panel (CMP) test. The creatinine, or serum creatinine, levels may be input in units of milligrams per decilitre (mg/dL) and may be obtained from a creatinine blood test. The ammonia level may be input in the units of micromole per litre of blood (μmol/L) or may be converted to a calibrated ammonia level, such as ammonia level normalised to upper limit of normal (AMM-ULN) before input.

The use of AMM-ULN is a concept that was recently developed and validated (see, for example reference [13]) and harmonises ammonia measurements across, for example, test centres. The inventors found that using ammonia levels by themselves may be ineffective. Measured ammonia levels are dependent on, for instance, the calibration of the test device taking the measurement and/or the timeframe over which a, for example, blood sample is measured. The inventors found that using an AMM-ULN level standardised the ammonia measured for all patients within each hospital. An upper limit of normal may be taken for each hospital and used to calculate an AMM-ULN value for all patients belonging to each hospital.

The method may include converting an input ammonia level into an AMM-ULN ratio by dividing the input level by a reference laboratory upper limit of normal for ammonia [(AMM-ULN)=plasma ammonia (μmol/L)/upper limit of normal for ammonia (μmol/L) in each individual laboratory]. The subject's ammonia level (or serum ammonia level) may be obtained from a measurement of ammonia concentration in a blood sample.

In step 20 of the method, the mathematical model assesses the probability of the subject developing OHE over time. For example, a random forest ML model may be obtained as explained in more detail later, by growing e.g. 500 trees using training data. The 5 predictor variables of the subject may be fed into each tree of the ML model and evaluated at each node until they reach a terminal node (or leaf).

Each terminal of each tree may be assigned a survival curve calculated from, for example, a survival function using the Kaplan-Meier estimator. An ensemble survival function may be obtained by taking the average of the survival functions of each node at which the input variables (i.e., the subject) have terminated: these may be referred to as subject terminal nodes. The machine model may use the ensemble survival curve, which may be in the form of a Kaplan-Meier curve, to assess the probability of the subject developing OHE based on the five input predictor variables. Additionally or alternatively, the machine model may use the survival curve to predict the median survival time for a subject with the input variables.

The ML model may assess the probability of the subject developing OHE in a certain time frame by using the predictSurvProb from the pec package in R project. The function may take as an input three arguments: object, new data and time frame. The object is a fitted model from which to extract the predicted survival probabilities, for example an ensemble survival function of a random survival forest (an RF object). That is, the object may contain, all the survival functions for each terminal node of each tree in a random forest model. The new data may be the input variables of the subject and the time frame may be a vector of times, as defined in R project, over which to predict the survival probability. The resulting object may be used to produce a survival curve for a new subject. Survival curve visualization may be carried out with the ggplot function from the well-known ggplot2 package.

### Training Phase

The above machine model may be trained using training data. For example, a random survival forest may be trained using the variables: Sex, Diabetes Miletus status, Creatinine (mg/dL), AMM-ULN and Albumin (g/dL), along with time to develop OHE (i.e. time-to-event, TTE, and ‘Event or Censored’ data, that is if the event occurred in the duration of the study). The inventors used data from three hospitals: Hospital Clinico Universitario de Valencia, Virgen del Rocio Hospital and Royal Free London Hospital to train a random survival forest. 426 candidate subjects were initially identified but only complete cases, that is subjects with all 5 of the above variables, were used in the model. 40 patients were lost in this process and model was built on 386 patients. Of course, imputation may be used on incomplete data.

The function in R project used to train the model is rfsrc from the randomForestSRC package (Ishwaran et al., 2008; Ishwaran & Kolagur, 2007; Ishwaran & Kogalur, 2022). A formula is passed as main argument which may include time to OHE, the five variables and if the event occurred (i.e. if the event was censored). A second argument is the splitting rule used at each node. This may be, for example, a log-rank statistic.

In the random survival forest model, a bootstrap sample may be taken from the training data. The splitting rule and number of variables used in each split is determined and a tree is grown. 500 bootstrap samples may be taken from the training data and 500 trees may be grown. That is, 1 tree is grown per bootstrap sample. Approximately 63.2% of training data was used as in-bag data to train the model and 36.8% of the training data was used for out-of-bag validation. The number of variables used in the splitting may be determined by taking the closest number to the square root of the number of predictor variables. For example, in this method 5 predictor variables were used, and the number of variables used at each split was 2. Of course, any number of variables up to the total may be used. The inventors chose to use the log rank statistic as the splitting rule at each node.

The variables for each training subject in the training data were fed into the machine learning model. The gender of each subject was entered as either Male or Female and the diabetes status as True or False. The Albumin levels (i.e serum or blood albumin) were input in units of grams per decilitre (g/dL), the creatinine (or serum creatinine) levels were input in units of milligrams per decilitre (mg/dL) and the ammonia levels were converted to an ammonia level normalised to upper limit of normal (AMM-ULN) level and input. That is, an upper limit of normal was taken for each hospital and used to calculate an AMM-ULN value for all patients belonging to each hospital. Measured ammonia levels are dependent on, for instance, the calibration of the test device taking the measurement and/or the timeframe over which a, for example, blood sample is measured. The inventors found that using an AMM-ULN level standardised the ammonia measured for all patients within each hospital. The time to event, i.e. the time to develop OHE, was input in days and if the event occurred or was censored was input as a 1 for occurred and 0 for censored. Of course, the input variable may be in any units and the above are given by way of example only.

As will be appreciated by those skilled in the art, the machine learning model, for example a random forest model, may produce, as an output, a probability of a given subject developing OHE. For example, the training data at each terminal node may be used to non-parametrically estimate a survival and cumulative hazard function using the Kaplan-Meier and Nelson-Aalen estimators, respectively. Hence, each terminal node may be associated with a survival function and an ensemble survival function may be obtained by taking the average of the survival functions of each tree in the forest. The survival functions may be used by the machine model to assess the probability of a new subject developing OHE.

A validation dataset may be used to predict the prediction error of the machine learning model. As appreciated by those skilled in the art, an out-of-bag sample, that is the training data not selected in the bootstrap, may be used to validate the machine learning model. The inventors validated the random survival forest build using data from two independent cohorts: Kings College Hospital and the Medical University of Vienna (test cohorts). Descriptive analysis of these two cohorts can be found in Table 2. The pec function from the pec package in R project was used but in this case two more arguments passed to it: traindata and data. The first argument is the same for both test cohorts as it is set to the training cohort, that is the data used to train the machine learning model, while the second argument is set to the test cohort data being evaluated. Brier score, (BS) and integrated Brier Score (IBS) were used to evaluate performance. The BS is a weighted average of the squared distances between the observed survival status and the predicted survival probability of a model. The integrated Brier score calculates the predictive error over time with larger values of the Brier score indicating worse performance of the predictive model.

FIG. 1B shows a plot of number of trees in the random forest model against error rate. As described above, the inventors selected 500 trees for the random forest model. The inventors found that as the number of trees used in the model was increased the error rate, (e.g., the out-of-bag error rate or forest error rate) decreased. As shown in the Figure, the error rate began to stabilize below 0.310. 500 trees were selected for the model as the error rate was low, while the model remained computationally efficient.

### Evaluation of the ML Model

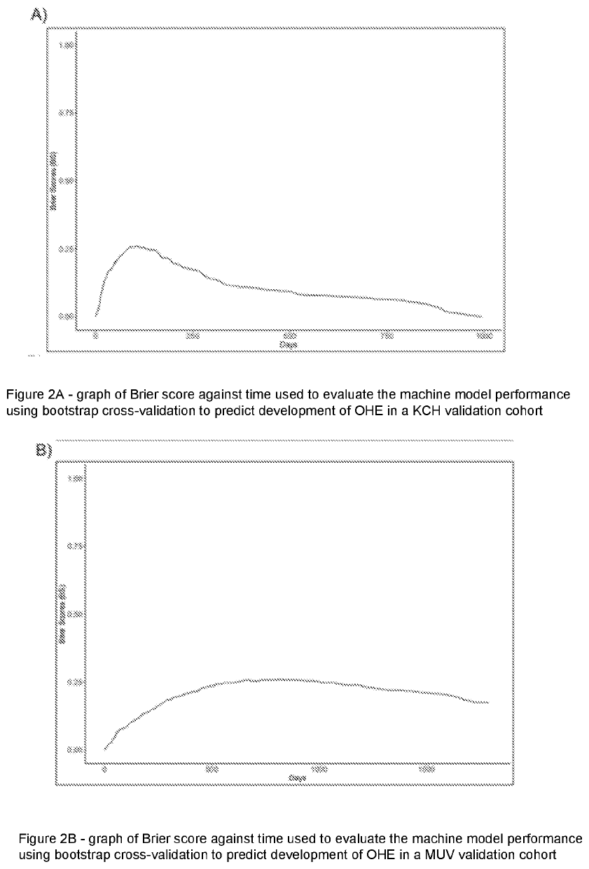

FIG. 2A shows the Brier score used to evaluate an AMMON-OHE model, performance using bootstrap cross-validation to predict development of OHE in the Kings College Hospital validation data. Application of the random forest model from the derivation sets (that is, the training data cohort) to the validation cohorts showed an integrated Brier Score of 0.137 for the KCH data set. Additionally, an estimate of prediction error was performed using Harrell's concordance index (C-index). The C-index of the AMMON-OHE model was 0.844 (standard error (se) 0.037) in the KCH dataset.

FIG. 2B shows the Brier score to evaluate the AMMON-OHE model performance using bootstrap cross-validation to predict development of OHE in the Medical University of Vienna test cohort. Application of the random forest model from the derivation sets to the validation cohorts showed an integrated Brier Score of 0.180 for the MUV dataset and gave a C-index of 0.728 (se 0.035).

### Comparative Methods

The prediction capabilities of the AMMON-OHE machine learning model were tested along with four other models commonly used to predict the onset of OHE: Child-Pugh score, MELD score, Psychometric hepatic encephalopathy score (PHES), and critical flicker frequency (CFF). A random survival forest was also tested using all independent variables given in Table 1 under laboratory parameters. The evaluation of the performance to predict risk of OHE in all six models was carried out using Brier score (BS) and integrated Brier score (IBS). To evaluate this performance pec function from the pec package (Mogensen, Ishwaran & Gerds, 2012) was used. All six model objects were included in a list as first argument, exact argument set as true, cens.model set as “marginal”, no reference and SplitMethod set to “Boot635plus” which is a Linear combination of AppErr and BootCv using weights dependent on how the models perform in permuted data. The number of bootstrap samples was set to default (100).

Cross-validation based on bootstrap resampling or bootstrap subsampling can be applied to assess and compare the predictive power of various regression modelling strategies on the same set of data. The inventors used cross-validation based on bootstrap resampling using the pec package “Boot632plus” which is a linear combination of “AppErr” (training error or apparent error obtained when the model(s) are evaluated in the same data where they were trained) and BootCv (the prediction models are trained on 100 bootstrap samples, that are either drawn with replacement of the same size as the original data. The models were assessed in the observations that are not in the bootstrap sample using weights dependent on how the models perform in permuted data.

TABLE 1
Patient characteristics according to psychometric test performance.

| | PHES | | | | | CFF | | | | | AMM_ULN | | | | |
|---|---|---|---|---|---|---|---|---|---|---|---|---|---|---|---|
| | Normal | | Abnormal | | p | Normal | | Abnormal | | p | Low | | High | | p |
| Parameter | (n = 252) | | (n = 174) | | value | (n = 216) | | (n = 109) | | value | (n = 212) | | (n = 214) | | value |
| Age (mean, SD) | 59 | (10) | 59 | (10) | 0.706 | 59 | (10) | 60 | (11) | 0.318 | 60 | (10) | 58 | (10) | 0.326 |
| Sex (no. male, %) | 172 | (68) | 116 | (67) | 0.731 | 146 | (68) | 70 | (64) | 0.544 | 157 | (74) | 131 | (61) | 0.005 |
| Disease Aetiology | | | | | | | | | | | | | | | |
| (Yes, %) | | | | | | | | | | | | | | | |
| ARLD | 114 | (45) | 89 | (51) | 0.230 | 100 | (46) | 57 | (52) | 0.308 | 95 | (45) | 108 | (51) | 0.273 |
| Viral Hepatitis | 100 | (40) | 63 | (36) | 0.469 | 87 | (40) | 34 | (41) | 0.110 | 88 | (41) | 75 | (35) | 0.170 |
| AILD | 12 | (5) | 10 | (6) | 0.652 | 8 | (4) | 8 | (70.8) | 0.153 | 10 | (5) | 12 | (55) | 0.678 |
| NAFLD | 13 | (6) | 8 | (5) | 0.793 | 12 | (6) | 6 | (5) | 0.985 | 12 | (6) | 9 | (4) | 0.489 |
| Other | 13 | (5) | 4 | (3) | 0.139 | 9 | (4) | 4 | (4) | 0.829 | 7 | (3) | 10 | (5) | 0.470 |
| Diabetes mellitus | 88 | (35) | 66 | (38) | 0.545 | 73 | (34) | 47 | (43) | 0.107 | 74 | (35) | 80 | (38) | 0.570 |
| Laboratory | | | | | | | | | | | | | | | |
| Parameters | | | | | | | | | | | | | | | |
| (mean, SD) | | | | | | | | | | | | | | | |
| AST (iU/mL) | 50.3 | (34.6) | 47.9 | (30.2) | 0.508 | 43.2 | (25.3) | 44.6 | (26.5) | 0.674 | 46.4 | (33.2) | 52.7 | (32.3) | 0.388 |
| ALT (iU/mL) | 40.5 | (34.1) | 47.3 | (38.3) | 0.077 | 41.2 | (37.9) | 41.6 | (25.5) | 0.925 | 39.0 | (33.9) | 48.2 | (37.8) | 0.192 |
| Albumin (g/dL) | 3.7 | (0.8) | 3.3 | (0.7) | <0.001 | 13.7 | (0.7) | 3.5 | (0.8) | 0.044 | 4.0 | (0.7) | 3.1 | (0.6) | 0.823 |
| Bilirubin (mg/dL) | 2.3 | (3.8) | 3.2 | (4.2) | 0.030 | 2.3 | (3.8) | 2.6 | (3.4) | 0.514 | 1.5 | (2.7) | 3.8 | (4.6) | <0.001 |
| Creatinine (mg/dL) | 0.9 | (0.4) | 0.9 | (0.5) | 0.682 | 0.9 | (0.4) | 0.9 | (0.4) | 0.603 | 0.9 | (0.3) | 1.0 | (0.5) | <0.001 |
| INR | 1.3 | (0.3) | 1.4 | (0.3) | 0.016 | 1.2 | (0.3) | 1.3 | (0.4) | 0.004 | 1.2 | (0.3) | 1.4 | (0.3) | <0.001 |
| Sodium (mmol/L) | 138.3 | (4.1) | 137.1 | (4.4) | 0.015 | 139.0 | (3.7) | 137.4 | (3.9) | 0.003 | 139.2 | (3.2) | 136.5 | (4.8) | <0.001 |
| AMM-ULN | 0.9 | (0.7) | 1.3 | (0.7) | <0.001 | 1.01 | (0.8) | 1.05 | (0.6) | 0.658 | 0.5 | (0.3) | 1.6 | (0.6) | <0.001 |
| Child Pugh Score | 7 | (2) | 8 | (2) | 0.004 | 7 | (2) | 7 | (2) | 0.094 | 6 | (2) | 8 | (2) | <0.001 |
| (mean, SD and | | | | | | | | | | | | | | | |
| n, %) | | | | | | | | | | | | | | | |
| A | 85 | (49) | 38 | (30) | <0.001 | 79 | (50) | 24 | (36) | 0.057 | 81 | (68) | 42 | (23) | <0.001 |
| B | 57 | (33) | 66 | (50) | 0.002 | 57 | (36) | 29 | (44) | 0.286 | 28 | (24) | 95 | (51) | <0.001 |
| C | 30 | (17) | 27 | (21) | 0.456 | 20 | (13) | 12 | (66) | 0.291 | 8 | (7) | 49 | (26) | <0.001 |
| MELD (mean, SD) | 11 | (5) | 12 | (6) | 0.027 | 11 | (6) | 11 | (6) | 0.372 | 10 | (4) | 13 | (6) | <0.001 |
| Compensated (n, %) | 141 | (56) | 63 | (36) | <0.001 | 131 | (60) | 40 | (37) | <0.001 | 144 | (68) | 60 | (28) | <0.001 |
| Development of 1- | 19 | (8) | 20 | (12) | 0.165 | 15 | (7) | 13 | (12) | 0.131 | 14 | (7) | 26 | (12) | 0.050 |
| year OHE (n, %) | | | | | | | | | | | | | | | |
| Transplant (n, %) | 17 | (7) | 8 | (5) | 0.354 | 14 | (6) | 6 | (7) | 0.730 | 15 | (7) | 10 | (5) | 0.292 |
| Mortality at 1-year | 17 | (7) | 22 | (13) | 0.032 | 21 | (10) | 11 | (10) | 0.205 | 9 | (4) | 30 | (14) | 0.611 |
| (n, %) | | | | | | | | | | | | | | | |

Comparisons were performed between normal and abnormal psychometric performance using t-test for continuous data and Chi-Square (χ²) for categorical data.
Abbreviations: ARLD, alcohol-related liver disease; AILD, autoimmune liver disease; NAFLD, non-alcoholic fatty liver disease; AST, aspartate aminotransferase; ALT, alanine aminotransferase; INR, international normalised ratio; AMM-ULN, ammonia upper limit of normal; MELD, model for end-stage liver disease score; PHES, psychometric hepatic encephalopathy score; CFF, critical flicker frequency; OHE, overt hepatic encephalopathy; SD, standard deviation.

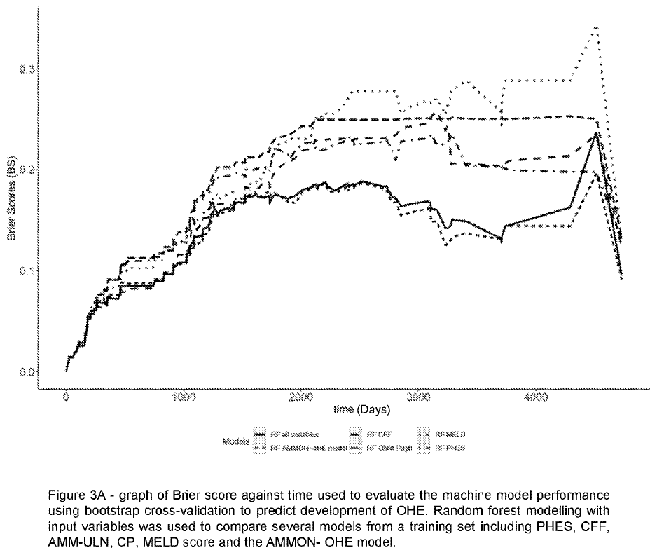

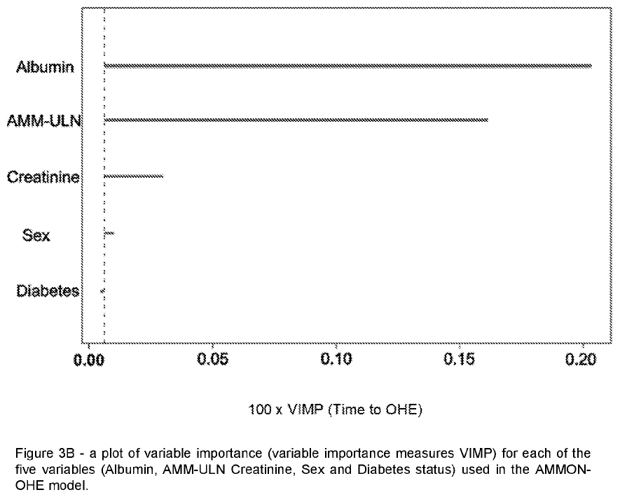

FIG. 3A shows a graph of Brier score against time for the 6 test models: AMMON-OHE machine learning model, Child-Pugh score, MELD score, Psychometric hepatic encephalopathy score (PHES), critical flicker frequency (CFF) and a machine learning model using all variables. Fast unified random forests for survival with 500 trees using log-rank as splitting criteria was used to predict the risk of the episode (or first episode) of OHE using bootstrapping and cross-validation in the combined dataset. Validation of the random forest was carried out using Brier score and integrated Brier score. Application of the random forest model showed that the best model to predict development of OHE was the AMMON-OHE model with the lowest integrated Brier score (FIG. 3A and Table 1.1 below).

Table 1.1 shows the integrated brier score for the risk of development of OHE for each of the variables shown in FIG. 3A. The AMMON-OHE model has a score of (0.148). While this score is slightly higher than the model with all variables (0.144), the inventors found that the ‘all variables’ model may be too complex to be applied in a clinical setting. For example, the ‘all variables’ model is computationally expensive to train, and it is often difficult to obtain complete data (i.e. all parameters in Table 1) for each patient. As shown in Table 1.1, the AMMON-OHE model outperformed the remaining models.

TABLE 1.1
Integrated Brier Score for the risk of development of OHE.

| Variable | Integrated Brier Score |
|---|---|
| All variables | 0.144 |
| AMMON-OHE model | 0.148 |
| PHES | 0.187 |
| CFF | 0.199 |
| MELD | 0.161 |
| Child-Pugh | 0.156 |
| MELD-sex-diabetes-albumin-creatinine | 0.158 |
| Child-Pugh-sex-diabetes-creatinine | 0.152 |

To develop the random survival forest model the inventors identified suitable variables to use as training data. The first step for its development was the use of univariable and multivariable competing frailty risk analysis using data from the three training data hospitals which were included as the frailty term: Hospital Clinico Universitario de Valencia, Virgen del Rocio Hospital and Royal Free London Hospital. This analysis was performed in order to assess which combination of variables had the lowest Akaike's Information Criterion (AIC). Included variables where: Age, Sex, Albumin, Br (Bilirubin), INR (international normalized ratio), Creatinine, Sodium, Ammonia upper limit of normal (AMM-ULN), Diabetes Miletus status, MELD (Model for End Stage Liver Disease) score and Child-Pugh score. The best model according to AIC included Sex, Diabetes Miletus status, Creatinine (mg/dL), AMM-ULN and Albumin (g/dL). In this first step, R project packages used were: survminer (Kassambara & Biecek, 2021) and survival (Therneau, 2022; Therneau & Grambsch, 2000). Descriptive analysis of these and other variables can be found in Table 2.

TABLE 2
Patient characteristics in the three prospective cohorts.

| | Total | HCUV | RFH | VRH | |
|---|---|---|---|---|---|
| Parameter | (n = 426) | (n = 156) | (n = 150) | (n = 120) | p value |
| Age (mean, SD) | 59 (10) | 62 (9) | 57 (11) | 58 (10) | <0.001 |
| Sex (no. male, %) | 288 (68) | 125 (80) | 79 (53) | 84 (70) | <0.001 |
| Disease Aetiology (n, %) | | | | | 0.007 |
| ARLD | 203 (48) | 69 (44) | 73 (49) | 61 (51) | |
| Viral Hepatitis | 162 (38) | 58 (36) | 59 (39) | 46 (38) | |
| AILD | 22 (5) | 4 (3) | 9 (6) | 9 (2) | |
| NAFLD | 21 (5) | 14 (9) | 3 (2) | 4 (3) | |
| Other | 17 (4) | 11 (7) | 6 (4) | 0 (0) | |
| Diabetes mellitus | 154 (36) | 55 (36) | 55 (37) | 44 (37) | 0.971 |
| Laboratory Parameters | | | | | |
| (mean, SD) | | | | | |
| AST (iU/mL) | 49 (33) | 43 (23) | 56 (37) | 52 (38) | 0.005 |
| ALT (iU/mL) | 43 (36) | 33 (19) | 59 (46) | 43 (38) | <0.001 |
| Albumin (g/dL) | 3.5 (0.8) | 3.9 (0.6) | 2.9 (0.6) | 3.9 (0.7) | <0.001 |
| Bilirubin (mg/dL) | 2.7 (3.9) | 1.3 (1.2) | 4.9 (5.7) | 1.6 (1.2) | <0.001 |
| Creatinine (mg/dL) | 0.9 (0.4) | 0.9 (0.3) | 1.04 (0.6) | 0.9 (0.2) | <0.001 |
| INR | 1.3 (0.3) | 1.2 (0.3) | 1.5 (0.3) | 1.2 (0.3) | <0.001 |
| Leucocytes (×10⁹/L) | 6.0 (3) | 5.9 (2) | 6.6 (4) | 5.6 (2) | 0.041 |
| Sodium (mmol/L) | 138 (4) | 139 (3) | 135 (5) | 140 (3) | <0.001 |
| AMM-ULN | 1.1 (0.7) | 0.9 (0.9) | 1.5 (0.4) | 0.8 (0.4) | <0.001 |
| Ammonia (μmol/L) | 56 (33) | 29 (29) | 61 (17) | 67 (35) | <0.001 |
| Child Pugh Score (mean, SD | 7 (2) | 6 (1) | 9 (2) | 6 (1) | <0.001 |
| and n, %) | | | | | |
| A | 203 (48) | 108 (69) | 16 (11) | 79 (66) | |
| B | 162 (38) | 43 (28) | 82 (55) | 37 (31) | <0.001 |
| C | 61 (14) | 5 (3) | 52 (35) | 4 (3) | |
| MELD (mean, SD) | 11 (6) | 10 (4) | 14 (7) | 10 (3) | <0.001 |
| Compensated (n, %) | 204 (48) | 131 (84) | 24 (16) | 49 (41) | <0.001 |
| Abnormal PHES (n, %) | 174 (41) | 43 (28) | 88 (59) | 43 (36) | <0.001 |
| Abnormal CFF (n, %) | 109/325 (34) | 32/137 (21) | 34/86 (23) | 43/102 (36) | 0.004 |
| Development of OHE during | 79 (19) | 22 (14) | 16 (11) | 41 (52) | <0.001 |
| follow-up (n, %) | | | | | |
| Development of 1-year OHE | 40 (9) | 8 (5) | 14 (9) | 18 (15) | 0.021 |
| (n, %) | | | | | |
| Mortality (n, %) | 105 (25) | 40 (26) | 29 (19) | 36 (30) | 0.122 |
| Mortality at 1-year (n, %) | 39 (9) | 9 (6) | 24 (16) | 6 (5) | <0.001 |
| Follow-up, months (median, | 30 (1-166) | 46 (1-81) | 8 (1-15) | 62 (1-166) | <0.001 |
| range) | | | | | |

Comparisons were performed between patient cohorts using ANOVA for continuous data and Chi-Square (χ²) for categorical data. Abbreviations: HCUV, Hospital Clinico Universitario de Valencia; RFH, Royal Free Hospital; VRH, Virgen del Rocio Hospital; ARLD, alcohol-related liver disease; AILD, autoimmune liver disease; NAFLD, non-alcoholic fatty liver disease; AST, aspartate aminotransferase; ALT, alanine aminotransferase; INR, international normalised ratio; AMM-ULN, ammonia upper limit of normal; MELD, model for end-stage liver disease score; PHES, pshychometric hepatic encephalopathy score; CFF, critical flicker frequency; OHE, overt hepatic encephalopathy; SD, standard deviation.

Comparisons between hospitals were performed using ANOVA or Kruskal Wallis for normally and non-normally distributed quantitative data, respectively. Categorical data were reported as number and percentage (%) and comparisons analyzed by Chi-squared (χ²) test.

To identify factors independently associated with OHE in the training set (n=426), univariable and multivariable competing risk frailty analyses using Fine-Gray sub-distribution hazard modelling were performed, considering liver transplantation as a competing event. The AMMON-OHE model was developed including only original variables to avoid multicollinearity. Thus, the variables selected for the AMMON-OHE model were Sex, Diabetes Miletus status, Creatinine (mg/dL), AMM-ULN and Albumin (g/dL) levels. Akaike Information Criterion (AIC) was used to select the best model, i.e., the model with the above 5 variables. AIC was also used to compare the machine learning model against PHES, CFF, AMM-ULN alone, their combination, and traditional severity scores (Model for End Stage Liver Disease (MELD) and Child Pugh (CP)). Validation of competing risk models was assessed using C-index.

All analyses were performed with R (version 4.0.2, R Core, 2021) with the cut-off for statistical significance set at 0.05. Survival package [18] was used to calculate univariable and multivariable competing risk frailty models and randomForestSRC package [19] for the random forest analyses and pec package [20] for Brier score model comparison.

FIG. 3B shows a plot of variable importance (variable importance measures VIMP) for each of the five variables identified for the AMMON-OHE model. As described above, factors independently associated with OHE in the training set were identified using univariable and multivariable analyses.

The importance, or impact, of each variable in the model was investigated using a variable importance measure (VIMP). Types of VIMP which may be used are Mean Decrease Impurity (MDI), Mean Decrease Accuracy (MDA) and the Sobol-MDA algorithm. The confidence intervals shown in Figure were constructed using subsampling.

As shown in FIG. 3B, Ammonia upper limit of normal (AMM-ULN) and Albumin were identified as having the highest variable importance, exceeding creatinine, sex and diabetes. While all 5 variables contribute to the accuracy of the model, the two variables providing the highest contribution are ammonia and albumin. Thus, to assess and output a risk of a subject developing OHE, the mathematical model (e.g. the random forest model) may take as an input blood AMM-ULN and blood Albumin levels and as a further optional input creatinine level, sex and diabetes status.

### Development and Testing of the ML Model

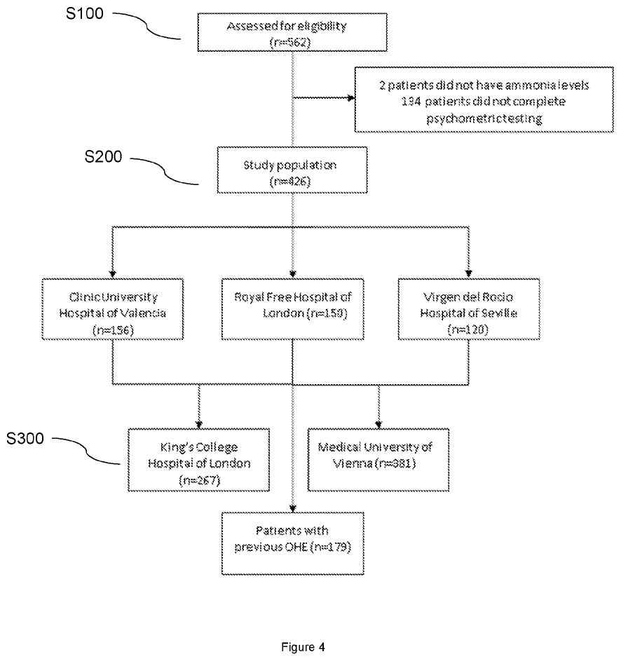

FIG. 4 shows a flowchart of the selection of the study population used in the training and validation of the AMMON-OHE machine learning model. The inventors hypothesized that ammonia levels may improve the ability of or even outperform currently used neuropsychological or psychophysical tests to stratify the risk of development of OHE in outpatients with cirrhosis. Therefore, they aimed to determine the role of ammonia and neuropsychological or psychophysical tests and develop a new prognostic model, the AMMON-OHE model, to stratify the risk of development of the episode (or first episode) of OHE in stable outpatients with cirrhosis. Secondary aims were to assess the utility of ammonia, neuropsychological or psychophysical test performance and the newly derived AMMON-OHE model for stratifying mortality risk and for predicting recurrent OHE in patients with a previous episode. The inventors tested the machine learning model against PHES and CFF tests. The PHES test is the accepted “gold standard” by the main liver societies, while CFF was selected as the guidelines recommend using two tests that evaluate different aspects of cognitive function. PHES and CFF are neuropsychological and psychophysical tests, respectively.

The AMMON consortium was created to determine the role of ammonia in the pathogenesis and treatment of complications of cirrhosis. S100 shows a step in which subjects were assessed for eligibility in the study. A prospective observational study of outpatients with cirrhosis from the AMMON cohort followed-up in three independent tertiary hospitals in Europe (Clinic University Hospital of Valencia, Spain (HCUV), n=156; Royal Free Hospital of London, United Kingdom (RFH), n=150; and Virgen del Rocio Hospital of Seville, Spain, (VRH) n=120) was conducted. Inclusion criteria were age ≥18 years and cirrhosis based on liver histology or a combination of characteristic clinical, biochemical, and imaging features. Exclusion criteria were previous episodes of OHE, established neurological or psychiatric disorders and liver-related complications (including new-onset ascites, variceal bleeding or infection requiring antibiotics) within the past 4 weeks. Of course, the inclusion and exclusion criterion could be altered.

S200 shows a step in which 426 eligible patients with selected as the study population. All patients underwent neuropsychological or psychophysical testing and venous ammonia measurements on the same day. The psychometric hepatic encephalopathy score (PHES) and the critical flicker frequency (CFF) test were used to evaluate cognitive function [21]. PHES was performed by all patients and most of the patients (76%) also underwent CFF in the three study centres. Psychometric hepatic encephalopathy score (PHES)<−4 or critical flicker frequency (CFF)<39 was considered abnormal. Venous ammonia levels were measured in each of the hospitals using a standard operating procedure that involved collection of the blood sample in cooled EDTA tubes, rapid sample transport to the laboratory on ice and spectrophotometric assays. Ammonia was normalized to upper limit of normal (AMM-ULN) at the respective reference laboratory. Patients were followed until transplantation, death, or study closure (in which case the data was censored). The outpatients were followed for a median of 2.5 years.

Further Information collected at the time of inclusion were age, sex, aetiology of cirrhosis, presence of type 2 diabetes mellitus, compensated or decompensated stage of cirrhosis, disease severity assessed by Model for End Stage Liver Disease (MELD) and Child Pugh (CP) scores and laboratory parameters[16-17]. A PHES <−4 or a CFF test <39 Hz were considered abnormal [21].

As the normal range for ammonia varied across centres, plasma levels were adjusted to the individual laboratory's upper limit of normal [(AMM-ULN)=plasma ammonia (μmol/L)/upper limit of normal for ammonia (μmol/L) in each individual laboratory][22]. AMM-ULN was considered high when it was >1. The main outcomes of the study were development of a an episode (or first episode) of OHE requiring hospitalization (West Haven ≥2 [7,23) and mortality during follow-up.

S300 shows that two cohorts of patients from two independent liver units (King's College Hospital, London, United Kingdom (KCH), n=267 patients; and Medical University of Vienna, Vienna, Austria (MUV), n=381) with the same inclusion and exclusion criteria were used for external validation of the model. An additional cohort of outpatients with cirrhosis and previous episodes of OHE from KCH (n=179) was included to evaluate the utility of the AMMON-OHE for predicting recurrent OHE.

The study was approved by ethical review boards at all study sites.

### Results

### Study Population

As mentioned above, a total of 426 patients (or subjects) were included in the study, with 386 subjects' data used to train the machine model. Patient characteristics describing the individual cohorts are summarized in Table 2. Compared with HCUV and VRH patients, the RFH cohort presented more advanced liver disease with higher CP and MELD (ANOVA p<0.001) and higher AMM-ULN (ANOVA p<0.001). Median follow-up of the total cohort was 30 months (range 1-166 months).

Overall, 41% and 34% of patients had abnormal PHES or CFF, respectively. Forty three percent of the 174 patients with abnormal PHES had abnormal CFF; whilst only 56% of the 109 patients with abnormal CFF had an altered PHES. The overall agreement between the two tests was 61%. Patient characteristics according to test performance are summarized in Table 1. Compared with the group of patients with normal PHES, patients with abnormal PHES had more severe liver disease with higher CP and MELD scores (p=0.004 and p=0.027, respectively) and AMM-ULN (p<0.001). Distribution of AMM-ULN based on clinical and biochemical variables is shown in Table 3.

TABLE 3
Distribution of AMM-ULN according to clinical variables

AMM-ULN

| Parameter | (mean, SD/r) | p value |
|---|---|---|
| Age | −0.062 | 0.202 |
| Sex | | 0.074 |
| Male | 1.0 (0.72) | |
| Female | 1.2 (0.68) | |
| Disease Aetiology | | |
| ARLD | 1.09 (0.7) | 0.574 |
| Viral Hepatitis | 1.01 (0.7) | 0.119 |
| Autoimmune liver disease | 1.21 (0.8) | 0.369 |
| NAFLD | 1.25 (1.1) | 0.259 |
| Other | 1.11 (0.6) | 0.868 |
| Diabetes mellitus | | 0.258 |
| Yes | 1.1 (0.7) | |
| No | 1.1 (0.7) | |
| Laboratory Parameters (mean, SD) | | |
| AST (iU/mL) | 0.121 | 0.022 |
| ALT (iU/mL) | 0.135 | 0.010 |
| Albumin (g/dL) | −0.447 | <0.001 |
| Bilirubin (mg/dL) | 0.265 | <0.001 |
| Creatinine (mg/dL) | 0.155 | 0.002 |
| INR | 0.278 | <0.001 |
| Sodium (mmol/L) | −0.229 | <0.001 |
| Child Pugh Score | 0.376 | <0.001 |
| A | 0.9 (0.9) | <0.001 |
| B | 1.4 (0.6) | <0.001 |
| C | 1.5 (0.5) | <0.001 |
| MELD | 0.316 | <0.001 |
| Compensated | | <0.001 |
| Yes | 0.8 (0.8) | |
| No | 1.3 (0.6) | |
| Development of OHE | | 0.105 |
| Yes | 1.2 (0.7) | |
| No | 1.0 (0.7) | |
| 1-year OHE | | 0.016 |
| Yes | 1.3 (0.7) | |
| No | 1.1 (0.7) | |
| Mortality | | 0.025 |
| Yes | 1.2 (0.8) | |
| No | 1.0 (0.7) | |
| 1-year mortality | | <0.001 |
| Yes | 1.6 (0.7) | |
| No | 1.0 (0.7) | |

Analysis of variance (ANOVA) and t-test were performed to compare ammonia distributions between categorical groups, Pearson rank correlations were used to calculate the correlation with continuous clinical variables. Abbreviations: ARLD, alcohol-related liver disease; NAFLD, non-alcoholic fatty liver disease; AST, aspartate aminotransferase; ALT, alanine aminotransferase; INR, international normalised ratio; AMM-ULN, ammonia upper limit of normal; MELD, model for end-stage liver disease score; SD, standard deviation; r, Pearson rank correlation coefficient.

### Risk Stratification for an Episode (or the First Episode) of OHE and Mortality According to PHES, CFF and Ammonia

Stratified groups according to PHES and CFF test results and AMM-ULN are described in Table 4 for PHES and Table 5 for CFF. The risk for hospitalization with OHE and mortality were significantly higher in patients with high AMM-ULN and either abnormal (HR (Hazard Ratio): 4.4, 95% Cl (confidence interval) 2.4-8.1; p<0.001 and HR: 5.1, 95% Cl 3.0-8.5; p<0.001, respectively) or normal PHES (HR: 2.2, 95% Cl 1.1-4.5; p=0.036 and HR: 2.9, 95% Cl 1.7-5.3; p<0.001, respectively) or either abnormal (HR: 5.6; 95% Cl 2.4-13.3; p<0.001 and HR: 4.7, 95% Cl 2.3-9.5; p<0.001, respectively) or normal CFF (HR: 4.2, 95% Cl 2.04-8.7; p<0.001 and HR: 3.6, 95% Cl 2.1-6.3; p<0.001, respectively) than in the low AMM-ULN and normal test groups (Table 6). Sub-analyses that included the interaction between risk groups and whether patients were compensated or decompensated were statistically non-significant (p=1.000), suggesting that risk stratification was maintained regardless of the stage of cirrhosis.

TABLE 4
Patient characteristics according to PHES and ammonia.

| | Low | Low | High | High | |
|---|---|---|---|---|---|
| | AMM-ULN/ | AMM-ULN/ | AMM-ULN/ | AMM-ULN/ | |
| | Normal PHES | Abnormal PHES | Normal PHES | Abnormal PHES | |
| Parameter | (n = 153) | (n = 59) | (n = 99) | (n = 115) | p value |
| Age (mean, SD) | 60 (10) | 61 (9) | 59 (10) | 58 (10) | 0.130 |
| Sex (no. male, %) | 117 (77) | 40 (68) | 55 (56) | 76 (66) | 0.014 |
| Disease Aetiology (Yes, %) | | | | | |
| ARLD | 65 (43) | 30 (51) | 49 (50) | 59 (51) | 0.152 |
| Viral Hepatitis | 69 (45) | 19 (32) | 31 (31) | 44 (38) | 0.159 |
| AILD | 5 (3) | 5 (9) | 7 (7) | 5(4) | 0.603 |
| NAFLD | 8 (5) | 4 (7) | 5 (5) | 4 (4) | 0.502 |
| Other | 6 (4) | 1 (2) | 7 (7) | 3 (3) | 0.996 |
| Diabetes mellitus | 50 (33) | 24 (41) | 38 (39) | 42 (37) | 0.480 |
| Laboratory Parameters (mean, SD) | | | | | |
| Albumin (g/dL) | 4.0 (0.6) | 3.8 (0.8) | 3.1 (0.7) | 3.1 (0.6) | <0.001 |
| Bilirubin (mg/dL) | 1.2 (1.4) | 2.2 (4.5) | 3.9 (5.2) | 3.7 (4.0) | <0.001 |
| Creatinine (mg/dL) | 0.9 (0.3) | 0.9 (0.3) | 1.0 (0.5) | 0.9 (0.5) | 0.059 |
| INR | 1.2 (0.2) | 1.2 (0.3) | 1.4 (0.4) | 1.4 (0.3) | <0.001 |
| Sodium (mmol/L) | 139.1 (3) | 139.4 (3) | 137.1 (4.9) | 136.0 (4.7) | <0.001 |
| AMM-ULN | 0.4 (0.2) | 0.6 (0.2) | 1.6 (0.6) | 1.6 (0.5) | <0.001 |
| Child Pugh Score (mean, SD | 6 (1) | 6 (2) | 8 (2) | 8 (2) | <0.001 |
| and n, %) | | | | | |
| A | 62 (69) | 19 (67) | 23 (27) | 19 (19) | <0.001 |
| B | 22 (24) | 6 (21) | 35 (42) | 60 (59) | <0.001 |
| C | 4 (4) | 4 (14) | 26 (31) | 23 (23) | <0.001 |
| MELD (mean, SD) | 9 (3) | 10 (5) | 14 (6) | 13 (6) | <0.001 |
| Compensated (n, %) | 106 (69) | 38 (64) | 35 (35) | 25 (22) | <0.001 |
| Development of OHE (n, %) | 26 (17) | 13 (22) | 12 (12) | 28 (24) | 0.314 |
| Development of 1 year OHE (n, %) | 10 (6.5) | 4 (6.8) | 9 (9) | 16 (14) | 0.041 |
| Transplant (n, %) | 10 (7) | 5 (9) | 7 (7) | 3 (3) | 0.218 |
| Mortality (n, %) | 28 (18) | 21 (36) | 23 (23) | 33 (29) | 0.106 |
| Mortality at 1 year (n, %) | 6 (4) | 3 (5) | 11 (11) | 19 (16) | 0.656 |

Comparisons were performed between the groups using ANOVA for continuous data and Chi-Square (χ²) for categorical data. Abbreviations: ARLD, alcohol-related liver disease; AILD, autoimmune liver disease; NAFLD, non-alcoholic fatty liver disease; AST, aspartate aminotransferase; ALT, alanine aminotransferase; INR, international normalised ratio; AMM-ULN, ammonia upper limit of normal; MELD, model for end-stage liver disease score; PHES, psychometric hepatic encephalopathy score; OHE, overt hepatic encephalopathy; SD, standard deviation.

TABLE 5
Patient characteristics according to CFF and ammonia.

| | Low | Low | High | High | |
|---|---|---|---|---|---|
| | AMM-ULN/ | AMM-ULN/ | AMM-ULN/ | AMM-ULN/ | |
| | Normal CFF | Abnormal CFF | Normal CFF | Abnormal CFF | |
| Parameter | (n = 123) | (n = 56) | (n = 93) | (n = 53) | p value |
| Age (mean, SD) | 60 (10) | 61 (10) | 58 (10) | 60 (12) | 0.039 |
| Sex (no. male, %) | 94 (76) | 39 (70) | 52 (56) | 31 (59) | 0.001 |
| Disease Aetiology (Yes, %) | | | | | |
| ARLD | 53 (43) | 26 (46) | 47 (51) | 31 (59) | 0.057 |
| Viral Hepatitis | 58 (48) | 19 (34) | 29 (31) | 15 (28) | 0.005 |
| Autoimmune liver disease | 2 (2) | 5 (9) | 6 (7) | 3 (6) | 0.154 |
| NAFLD | 7 (6) | 4 (7) | 5 (5) | 2 (4) | 0.634 |
| Other | 3 (2) | 2 (4) | 6 (7) | 2 (4) | 0.319 |
| Diabetes mellitus | 38 (31) | 22 (39) | 35 (38) | 25 (47) | 0.050 |
| Laboratory Parameters | | | | | |
| (mean, SD) | | | | | |
| AST (iU/mL) | 43 (27.8) | 46 (27.1) | 44 (20.3) | 43 (26.1) | 0.855 |
| ALT (iU/mL) | 37 (31.8) | 37 (24.5) | 49 (46.5) | 47 (25.9) | 0.095 |
| Albumin (g/dL) | 4.0 (0.6) | 4.0 (0.6) | 3.3 (0.6) | 3.0 (0.6) | <0.001 |
| Bilirubin (mg/dL) | 1.5 (3.2) | 1.2 (0.8) | 3.2 (4.3) | 3.7 (4.4) | <0.001 |
| Creatinine (mg/dL) | 0.9 (0.3) | 0.9 (0.3) | 1.0 (0.6) | 1.0 (0.6) | 0.012 |
| INR | 1.2 (0.2) | 1.2 (0.3) | 1.3 (0.3) | 1.5 (0.4) | <0.001 |
| Sodium (mmol/L) | 140 (2.8) | 139 (2.9) | 138 (4.7) | 136 (4.2) | <0.001 |
| AMM-ULN | 0.5 (0.2) | 0.5 (0.2) | 1.7 (0.7) | 1.6 (0.5) | <0.001 |
| Child Pugh Score | 6 (2) | 6 (1) | 8 (2) | 8 (2) | <0.001 |
| (mean, SD, n, %) | | | | | |
| A | 56 (70) | 18 (82) | 23 (30) | 6 (14) | <0.001 |
| B | 19 (24) | 3 (14) | 38 (49) | 26 (59) | <0.001 |
| C | 4 (5) | 0 (0) | 16 (21) | 12 (27) | <0.001 |
| MELD Score (mean, SD) | 9 (4) | 9 (3) | 13 (6) | 13 (7) | <0.001 |
| Compensated (n, %) | 96 (78) | 32 (57) | 35 (38) | 8 (15) | <0.001 |
| Development of OHE (n, %) | 17 (14) | 15 (27) | 22 (24) | 10 (19) | 0.214 |
| Development of 1 year OHE | 4 (3) | 5 (9) | 11 (12) | 8 (15) | 0.004 |
| (n, %) | | | | | |
| Transplant (n, %) | 8 (7) | 3 (5) | 6 (7) | 3 (6) | 0.890 |
| Mortality (n, %) | 21 (17) | 19 (34) | 30 (32) | 13 (25) | 0.078 |
| Mortality at 1 year (n, %) | 4 (12) | 4 (7) | 17 (18) | 7 (13) | 0.441 |

Comparisons were performed between the groups using ANOVA for continuous data and Chi-Square (χ²) for categorical data. Abbreviations: ARLD, alcohol-related liver disease; NAFLD, non-alcoholic fatty liver disease; AST, aspartate aminotransferase; ALT, alanine aminotransferase; INR, international normalised ratio; AMM-ULN, ammonia upper limit of normal; MELD, model for end-stage liver disease score; CFF, critical flicker frequency; OHE, overt hepatic encephalopathy; SD, standard deviation.

TABLE 6
Risk of hospitalization with OHE and mortality in the 4 groups
of study according to psychometric test and ammonia.

| | | |
|---|---|---|
| Groups of study | HR (95% CI) | p-value |

Development of OHE

| | | |
|---|---|---|
| Low AMM-ULN/Normal PHES | Ref. | Ref. |
| Low AMM-ULN/Abnormal PHES | 1.7 (0.8-3.5) | 0.178 |
| High AMM-ULN/Normal PHES | 2.2 (1.1-4.5) | 0.036 |
| High AMM-ULN/Abnormal PHES | 4.4 (2.4-8.1) | <0.001 |
| Low AMM-ULN/Normal CFF | Ref. | Ref. |
| Low AMM-ULN/Abnormal CFF | 2.6 (1.2-5.6) | 0.019 |
| High AMM-ULN/Normal CFF | 4.2 (2.04-8.7) | <0.001 |
| High AMM-ULN/Abnormal CFF | 5.6 (2.4-13.3) | <0.001 |

Survival

| | | |
|---|---|---|
| Low AMM-ULN/Normal PHES | Ref | Ref |
| Low AMM-ULN/Abnormal PHES | 2.4 (1.4-4.2) | 0.003 |
| High AMM-ULN/Normal PHES | 2.9 (1.7-5.3) | <0.001 |
| High AMM-ULN/Abnormal PHES | 5.1 (3.01-8.5) | <0.001 |
| Low AMM-ULN/Normal CFF | Ref | Ref |
| Low AMM-ULN/Abnormal CFF | 1.97 (1.1-3.7) | 0.032 |
| High AMM-ULN/Normal CFF | 3.6 (2.1-6.3) | <0.001 |
| High AMM-ULN/Abnormal CFF | 4.7 (2.3-9.5) | <0.001 |

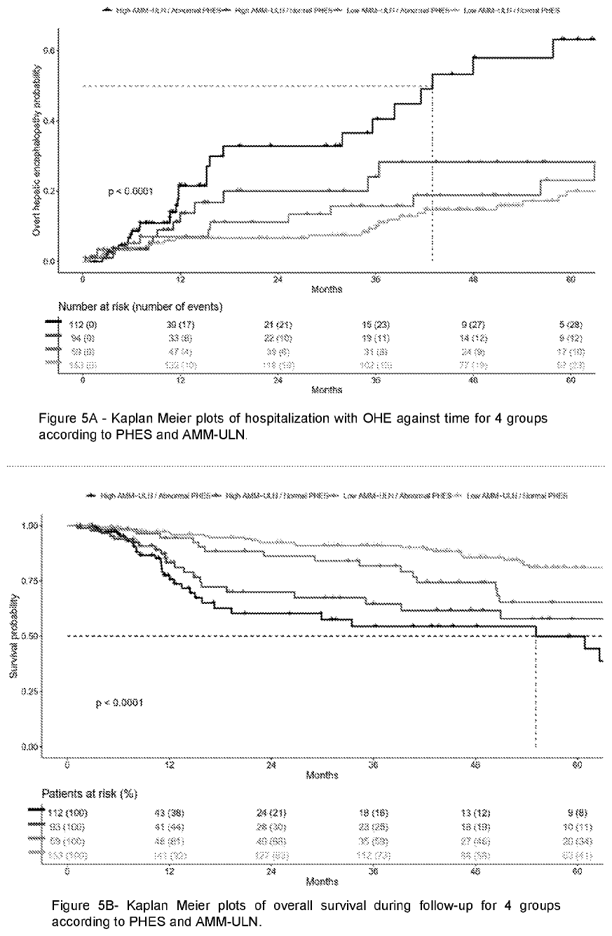

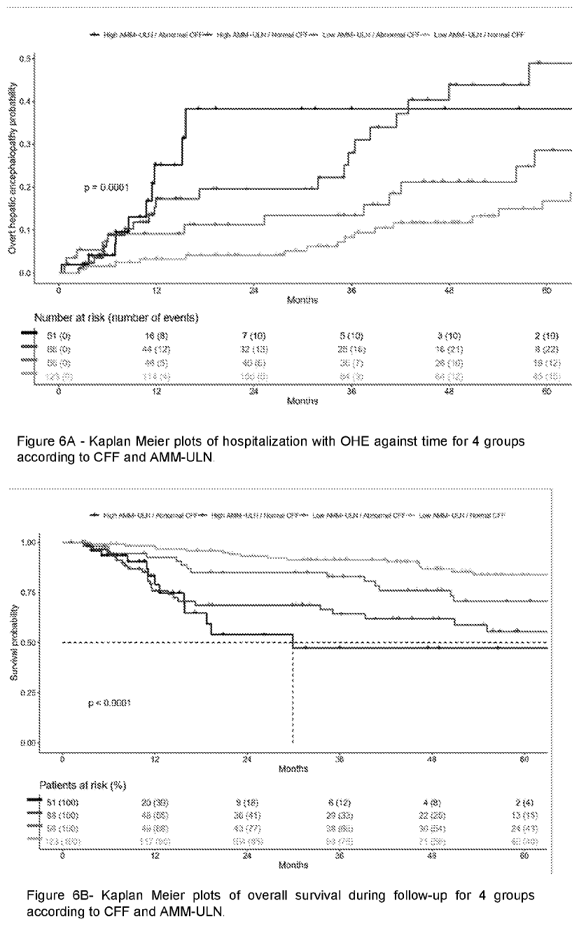

FIG. 5a shows Kaplan Meier plots of hospitalization with OHE in the above 4 stratified groups according to PHES and AMM-ULN. That is, for PHES and CFF (see FIGS. 6A and 6B), patients were stratified into four groups according to test performance and ammonia levels: 1) normal test-normal AMM-ULN, 2) abnormal test-normal AMM-ULN, 3) normal test-high AMM-ULN, and 4) abnormal test-high AMM-ULN. Kaplan-Meier curves were constructed to evaluate time to development of OHE and mortality. Differences in survival were assessed using log-rank tests. Pairwise comparisons between groups were adjusted for multiplicity using Benjamini & Hochberg method [24].

The Kaplan Meier plots demonstrate the cumulative probability for development of OHE. Differences in median overall survival were assessed by log-rank test. Cumulative probability of developing OHE at 1, 2, 3, 4 and 5 years was 0.07, 0.08, 0.10, 0.15 and 0.20 in the low AMM-ULN plus normal PHES group; 0.07, 0.11, 0.16, 0.19 and 0.23 in the low AMM-ULN plus abnormal PHES group; 0.14, 0.20 and 0.24 at 1, 2 and 3 years (no further cases) in the high AMM-ULN plus normal PHES group; and 0.22, 0.33, 0.41, 0.53 and 0.63 at 1, 2, 3, 4 and 5 years in the high AMM-ULN plus abnormal PHES group.

FIG. 5A shows that significant differences may be observed between the 4 groups for development of an episode (or first episode) of OHE (log-rank p<0.001). Multiple comparison with correction of p-values showed significant differences between high AMM-ULN and abnormal PHES with both groups of low AMM-ULN (abnormal PHES: p=0.008 and normal PHES: p<0.001), but no difference was observed between the two high AMM-ULN groups according to PHES performance (p=0.054).

Significant differences were also observed between the 4 study groups for overall survival (log-rank p<0.001). FIG. 5B shows a Kaplan Meier plot demonstrating cumulative probability for overall survival during follow-up. Similarly, multiple comparisons showed significant differences between high AMM-ULN-abnormal PHES and low AMM-ULN groups (p=0.007 compared with abnormal PHES and p<0.001 compared with normal PHES) in survival but no difference was observed compared with high AMM-ULN and normal PHES (p=0.189).

FIGS. 6A and 6B show Kaplan Meier plots of hospitalization with OHE and mortality in the 4 groups according to CFF and AMM_ULN, respectively. The Kaplan Meier plots demonstrate cumulative probability of the development of A) OHE and B) overall survival during follow-up. Differences in median overall survival were assessed by log-rank test.

Focusing on risk stratification according to CFF, significant differences were observed between the 4 study groups for development of an episode (or first episode) of OHE (log-rank p<0.001) (FIG. 6A) and overall survival (log-rank p<0.001) (FIG. 6B).

The inventors found significant differences in time-to-OHE (log-rank p<0.001) according to PHES and CFF tests and ammonia, with the highest risk in patients with abnormal PHES plus high AMM-ULN (HR (hazard ratio): 4.4; 95% Cl: 2.4-8.1, p<0.001 compared with normal PHES and AMM-ULN). On multivariable analysis, AMM-ULN but not PHES or CFF was an independent predictor for development of OHE (HR: 1.4; 95% Cl: 1.1-1.9; p=0.015). The mentioned above AMMON-OHE machine learning model included sex, diabetes, albumin, creatinine and AMM-ULN, and showed a C-index of 0.844 and 0.728 in the two validation cohorts (KCH and MUV), respectively.

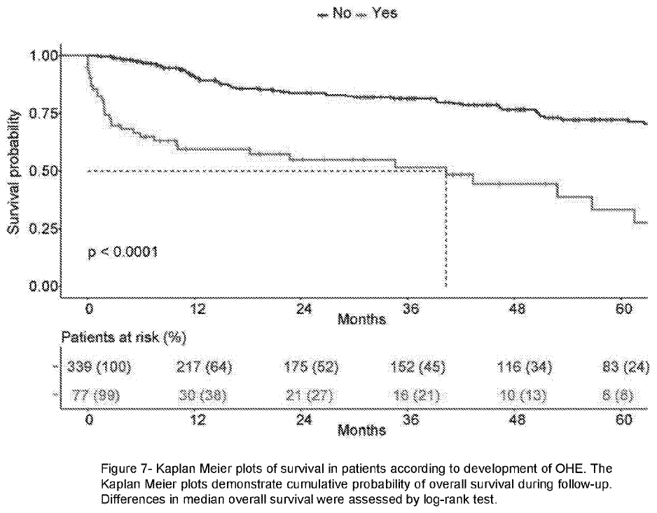

FIG. 7 shows Kaplan Meier plots of survival in patients according to development of OHE. The Kaplan Meier plots demonstrate cumulative probability of overall survival during follow-up. Differences in median overall survival were assessed by log-rank test. “Yes” in the key indicates the subjects which developed an episode (or first episode) of OHE during a follow-up while “No” indicates patients which did not developed OHE during follow-up. The inventors found that time to death was significantly shorter in patients who developed an episode (or first episode) of OHE compared with patients who did not develop OHE during follow-up (76±9 months vs 110±5 months; p<0.001; see FIG. 7) after study inclusion. Cumulative probability of death after an episode (or first episode) of OHE was 41% at 1 year.

### Prognostic Factors for Development of OHE and Mortality

The inventors found that abnormal PHES was a predictor for development of OHE in the univariable analysis (p=0.005) but not selected in the multivariable analyses including all variables, while CFF was not associated with OHE in the univariable analysis (p=0.130). High AMM-ULN was a risk factor for development of OHE in both the univariable (<0.001) and multivariable (p=0.015) analyses. The other independent risk factors for development of OHE on multivariable analysis were female sex (p=0.048) and lower albumin level (p<0.001) (Table 4). Although non-significant, diabetes mellitus (p=0.089) and creatinine level (p=0.130) were selected in the best multivariable competing risk frailty model to develop the AMMON-OHE model. Table 7 describes AIC of the PHES, CFF, AMM-ULN, their combination, CP, MELD, and the AMMON-OHE model. The AMMON-OHE model carried the lowest prediction error for the risk of an episode (or first episode) OHE. Application of the random forest model also showed that the best model to predict development of OHE was the AMMON-OHE model with the lowest integrated Brier score (FIG. 3A).

The AMMON-OHE model may qualify as a prognostic biomarker as it is able to identify the likelihood of an OHE event. The model may be used in clinical trials to set entry and exclusion criteria to identify higher-risk populations. In clinical trials, statistical power of a trial is often determined by number of events, instead of sample size, so using the model disclosed herein as a prognostic biomarker may increase event rates. Therefore, this biomarker may be used to determine treatment effect because the differences in outcomes as a function of treatment are magnified.

TABLE 7
Akaike Information Criterion (AIC) for the
risk of development of OHE and mortality.

| Parameter | AIC for OHE | AIC for mortality |
|---|---|---|
| PHES | 484.3 | 497.4 |
| CFF | 488.9 | 507.1 |
| AMM-ULN | 482.2 | 503.1 |
| PHES/AMM-ULN | 480.5 | 496.6 |
| CCF/AMM-ULN | 484.2 | 503.3 |
| MELD score | 487.8 | 501.7 |
| Child Pugh score | 480.0 | 501.2 |
| AMMON-OHE model | 474.8 | 494.5 |

Similarly, abnormal PHES was a predictor of mortality in the univariable frailty competing risk analysis (p<0.001) but not in the multivariable model, while an abnormal CFF result was not associated with survival (p=0.130). Again, higher AMM-ULN level was a risk factor for mortality in both the univariable (<0.001) and multivariable (p=0.008) analyses including all variables. Albumin was the other significant risk factor for mortality (p<0.001) and model performance was further improved with the inclusion of diabetes mellitus although it was not significant (Table 8). The AMMON-OHE model showed the best predictive accuracy for death compared with PHES, CFF, AMM-ULN, their combination, CP and MELD (Table 7).

TABLE 8
Univariable and multivariable model for risk of death.

| | Univariable | | Multivariable | |
|---|---|---|---|---|
| Parameter | HR (95% CI) | P value | HR (95% CI) | P value |
| Age | 1.04 (1.02-1.06) | <0.001 | | |
| Sex (female) | 1.01 (0.7-1.5) | 0.960 | | |
| Disease Aetiology | | | | |
| ARLD | Ref. | | | |
| Viral Hepatitis | 0.7 (0.5-1.1) | 0.130 | | |
| Autoimmune liver disease | 1.6 (0.8-3.2) | 0.180 | | |
| NAFLD | 0.7 (0.3-1.9) | 0.530 | | |
| Other | 1.8 (0.8-4.4) | 0.180 | | |
| Diabetes mellitus | 1.4 (0.9-1.9) | 0.098 | 1.3 (0.8-2.1) | 0.340 |
| Laboratory Parameters | | | | |
| Albumin | 0.4 (0.3-0.5) | <0.001 | 0.4 (0.2-0.5) | <0.001 |
| Bilirubin | 1.06 (1.03-1.1) | <0.001 | | |
| Creatinine | 1.3 (0.7-2.2) | 0.390 | | |
| INR | 1.8 (1.2-2.8) | 0.005 | | |
| Sodium | 0.93 (0.89-0.98) | 0.003 | | |
| AMM-ULN | 1.6 (1.3-2.002) | <0.001 | 1.6 (1.2-2.05) | 0.008 |
| Child Pugh Score | | | | |
| A | Ref. | | | |
| B | 2.4 (1.6-3.8) | <0.001 | | |
| C | 3.2 (1.6-6.1) | <0.001 | | |
| MELD | 1.06 (1.03-1.1) | <0.001 | | |
| Decompensated | 2.1 (1.9-3.6) | 0.009 | | |
| PHES (<−4) | 1.9 (1.3-2.9) | <0.001 | | |
| CFF (<39) | 1.4 (0.9-2.2) | 0.130 | | |

Variable selection for the multivariable competing risk frailty model was performed using stepwise forward-backward selection with estimated HR including only original variables within the final model. Abbreviations: ARLD, alcohol-related liver disease; NAFLD, non-alcoholic fatty liver disease; AST, aspartate aminotransferase; ALT, alanine aminotransferase; INR, international normalised ratio; AMM- ULN, ammonia upper limit of normal; MELD, model for end-stage liver disease score; HR, hazard ratio; CI, confidence interval.

### Validation of the AMMON-OHE Model and App Development for Risk of an Episode (or First Episode) of OHE

The inventors found that patients from KCH presented more severe disease with higher CP and MELD score than the derivation and Vienna cohorts (Table 9). The C-index of the AMMON-OHE model was 0.844 (standard error (se) 0.037) in KCH and 0.728 (se 0.035) in Vienna. C-index of MELD and CP scores were 0.695 (se 0.048) and 0.773 (se 0.030) and 0.613 (se 0.042) and 0.698 (se 0.036) in KCH and MUV, respectively, for the risk of future OHE.

Application of the random forest model from the derivation (training) sets to the validation cohorts showed an integrated Brier Score of 0.137 and 0.180 in KCH and MUV, respectively (FIG. 2).

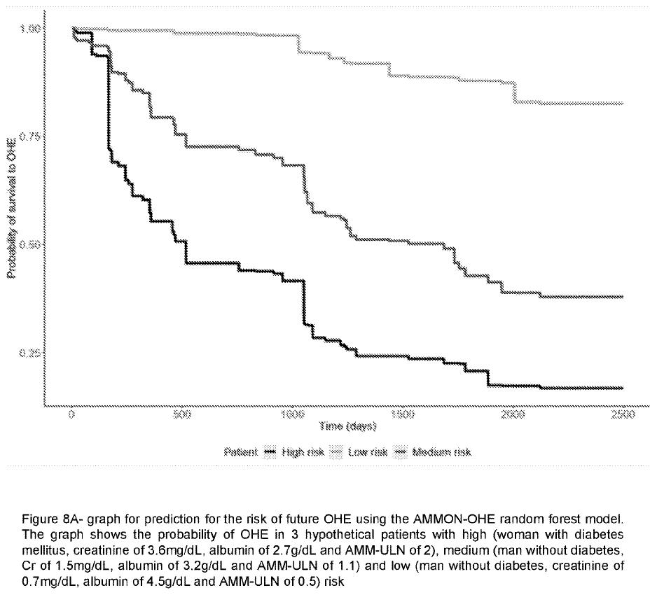

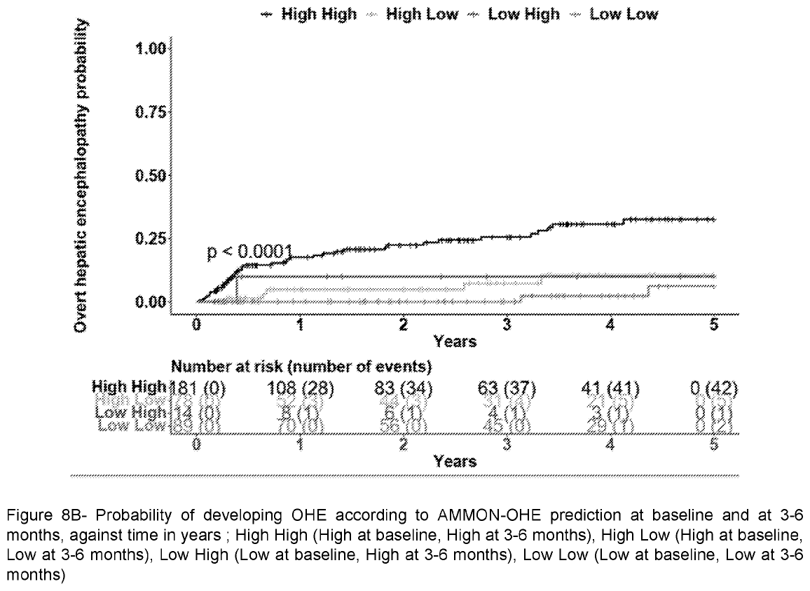

FIG. 8A shows a graph of probability of survival to OHE against Time (in days). The AMMON-OHE random forest model has been used to predict the risk of future OHE in 3 subjects. The figure shows the probability of OHE in 3 hypothetical patients with high (woman with diabetes mellitus, creatinine of 3.6 mg/dL, albumin of 2.7 g/dL and AMM-ULN of 2), medium (man without diabetes, Cr of 1.5 mg/dL, albumin of 3.2 g/dL and AMM-ULN of 1.1) and low (man without diabetes, creatinine of 0.7 mg/dL, albumin of 4.5 g/dL and AMM-ULN of 0.5) risk.

The machine model may predict the probability that each of the 3 patients develops OHE. The medium and/or high-risk patients may then be selected for therapy to, for example, prevent an episode of OHE. For instance, patients may be prescribed lactulose to take which is considered cheap and safe. Lactulose is a poorly absorbed disaccharide that works in several ways, for example by decreasing the blood ammonia concentration by promoting elimination of ammonia. Rifaximin is an alternative first-line treatment option for OHE or it is given as a concomitant therapy. It is a broad-spectrum antibiotic and it works by altering the bowel flora with net reduction in blood ammonia concentration. Of course, other drugs such as sodium benzoate, I-ornithine I-aspartate, I-ornithine phenyl acetate, glycerol phenylbutyrate, AST-120 (spherical carbon adsorbent), Branched-chain amino acids, lactitol, polyethylene glycol, Yaq-001 and/or VS-01 (Versantis/Genfit) may be prescribed and/or drugs and devices that modulate renal function such as terlipressin and midodrine may be used. Additional or alternative treatments may be increasing patient albumin levels by, for example, albumin infusion and/or nutritional support, devices or treat elevated creatinine and renal dysfunction using terlipressin or renal replacement therapy.

Currently, HE treatment is based on ammonia lowering drugs. The AMMON-OHE model may be used as a companion biomarker for both ammonia lowering drugs and other HE treatments, for example, the machine model may be used as a surrogate biomarker to assess response to therapy.

Assessment of each subject using the machine model could be done in repeated intervals. For example, in stable outpatients the repeated interval may be every 6 months. In acutely decompensated patients, it may be used every day during admission. As mentioned above, the model may risk stratify patients into low, medium and high risk groups. Patients with low risk may not need treatment, medium risk patients may require treatment on a case-by-case basis and it may be determined that high risk patients require treatment.

In order for clinicians and/or patients to easily use the AMMON-OHE model to predict, for example, future episodes of OHE in clinically stable outpatients or new patients or subjects, without any prior knowledge of machine learning, R project or programming, an app is provided. For example, such an app may be developed using Shiny (Chang et al., 2022). Shiny is an R package that provides a web application framework which can be self-hosted on the internet or in shinyapps.io. The open Source Shiny Server provides a platform on which one can host Shiny applications on a single server. It enables support for non-websocket-enabled browsers like Internet Explorer 10, and is available under an AGPLv3 license. The model and/or app may be used in everyday outpatient clinics and/or at the bed side in hospitalized patients, for example.

The input variables for the subject may be accessed from patient records, for example electronic patient records, EPRs. The inputs may be automatically fed into the model or input by a user.

### AMMON-OHE Model as Surrogate Biomarker

FIG. 8B shows a plot of probability of developing OHE according to the AMMON-OHE prediction at baseline and at 3-6 months. The AMMON-OHE model may be used as a surrogate biomarker, tracking the probability of developing OHE using repeat predictions.

In order to qualify as a surrogate biomarker, a correlation between AMMON-OHE and the outcome may be required. Furthermore, changes in the AMMON-OHE may be required to be related to the outcome i.e., developing or not developing OHE. In order to study the validity of the biomarker as a prognostic surrogate biomarker, a new external cohort was included with repeated measurements. This was an observational, single-center study of consecutive clinically stable outpatients with cirrhosis with at least two measurements of the AMMON-OHE model and no previous OHE episodes. Patients (N=362) were stratified into risk groups according to the AMMON-OHE at baseline. A change in the category was evaluated at 3-6 months and associated with the risk of OHE using Cox regression model and the Kaplan-Meier estimate.

FIG. 8B depicts the probability of OHE due to the changes in risk according to the AMMON-OHE model prediction at a 5-year timeframe. Statistical differences (log-rank test, p<0.001) were found between risk trajectories with patients showing a high-to-high-risk trajectory having the highest probability of developing OHE.

Patients that were high risk during both time points (i.e., High—High in the plot) had a significantly higher risk of developing OHE than patients that shifted to low risk (i.e., High—Low) or that were low during both timepoints (Low Low) (HR: 0.246, 95% Cl: 0.097-0.623, p=0.003 and HR: 0.077, 95% Cl: 0.019-0.320, p<0.001, respectively). No significant differences were found between high-risk patients at baseline and high risk during follow-up and low-risk patients at baseline and high-risk at follow-up.

TABLE 9
Patient characteristics in the training and validation cohorts.

| | Derivation | KCH | MUV | Prior OHE |
|---|---|---|---|---|
| Parameter | (n = 426) | (n = 267) | (n = 381) | (n = 179) |
| Age (mean, SD) | 59 (10) | 56 (10) | 55 (11) | 56 (10) |
| Sex (no. male, %) | 288 (68) | 171 (64) | 253 (66) | 122 (68) |
| Disease Aetiology (n, %) | | | | |
| ARLD | 203 (48) | 89 (33) | 153 (40) | 69 (38) |
| Viral Hepatitis | 162 (38) | 45 (17) | 140 (37) | 19 (11) |
| Autoimmune liver disease | 22 (5) | 66 (25) | 6 (2) | 31 (17) |
| NAFLD | 21 (5) | 44 (17) | 40 (11) | 45 (25) |
| Other | 17 (4) | 23 (9) | 42 (11) | 16 (9) |
| Diabetes mellitus, n (%) | 154 (36) | 74 (28) | 91 (24) | 79 (44) |
| Laboratory Parameters (mean, SD) | | | | |
| AST (iU/mL) | 49 (33) | 74 (62) | 62 (42) | 68 (57) |
| ALT (iU/mL) | 43 (36) | 52 (47) | 49 (48) | 43 (31) |
| Albumin (g/dL) | 3.5 (0.8) | 3.4 (0.6) | 3.6 (0.6) | 3.1 (0.5) |
| Bilirubin (mg/dL) | 2.7 (3.9) | 3.6 (4.6) | 1.7 (2.0) | 3.5 (4.0) |
| Creatinine (mg/dL) | 0.9 (0.4) | 0.9 (0.5) | 0.8 (0.3) | 0.9 (0.4) |
| INR | 1.3 (0.3) | 1.5 (0.5) | 1.4 (0.3) | 1.6 (0.5) |
| Leucocytes (×10⁹/L) | 6.0 (3) | 4.7 (2) | 5.1 (2.1) | 4.8 (2.6) |
| Sodium (mmol/L) | 138 (4) | 136 (5) | 138 (4) | 135 (4.6) |
| AMM-ULN | 1.1 (0.7) | 1.3 (0.6) | 0.8 (0.5) | 1.9 (1.0) |
| Child Pugh Score (mean, SD and n, %) | 7 (2) | 8 (2) | 7 (2) | 9 (2) |
| A | 203 (48) | 66 (25) | 224 (59) | 9 (5) |
| B | 162 (38) | 128 (48) | 134 (35) | 93 (52) |
| C | 61 (14) | 73 (27) | 23 (6) | 77 (43) |
| MELD (mean, SD) | 11 (6) | 14 (6) | 12 (4) | 15 (6) |
| Compensated (n, %) | 204 (48) | 96 (36) | 185 (49) | 19 (11) |
| Development of OHE (n, %) | 79 (19) | 33 (13) | 51 (13) | 49 (27) |
| Development of 1-year OHE (n, %) | 40 (9) | 30 (11) | 25 (7) | 49 (27) |
| Mortality (n, %) | 105 (25) | 25 (9) | 115 (30) | 26 (14) |
| Mortality at 1-year (n, %) | 39 (9) | 17 (6) | 26 (7) | 18 (10) |
| Transplant (n, %) | 25 (6) | 216 (81) | 28 (7) | 140 (78) |
| Follow-up, days (median, range) | 921 (34-5060) | 759 (2-1560) | 1049 (2-5820) | 770 (23-1849) |

Abbreviations: KCH, King's College Hospital; MUV, Medical University of Vienna; ARLD, alcohol- related liver disease; NAFLD, non-alcoholic fatty liver disease; AST, aspartate aminotransferase; ALT, alanine aminotransferase; INR, international normalised ratio; AMM-ULN, ammonia upper limit of normal; MELD, model for end-stage liver disease score; OHE, overt hepatic encephalopathy.

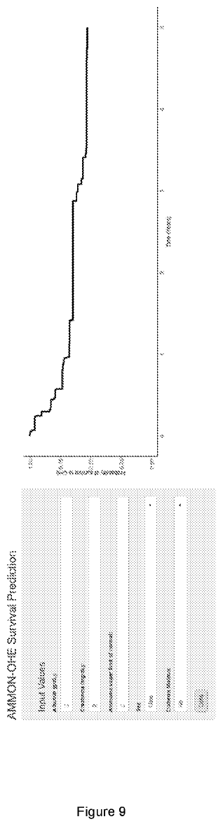

FIG. 9 shows an example of a graphical display on an interface which, for example, a clinician may interact with. The clinician may input the values of the 5 variables used in the machine model and click done. For example, on the left of the figure a table of input values is presented to a user. The app may then call the predictSurvProb function in R with the given input values, RF model object (that is, the machine learning model, and a time frame between, for example, 1 and 2500 days. The app may output a survival curve, for example a probability of developing OHE, and/or a table with estimated survival (time to event, i.e., time to develop OHE) at one, two, three, four and five years (in equal divisions along the defined time frame) as well as median survival time. The table may be displayed alongside the survival curve and/or may be displayed separately. As shown on the right of the figure, the app may output a Kaplan Meier curve for predicting probability of developing OHE over time. This graph may be displayed at the same time as the input variables table and/or output table and/or on a separate screen. Of course the output can be in any other suitable electronic form.

Prediction of OHE in Patients with and without History of OHE

Baseline characteristics of the cohort of patients with previous episodes of OH E are described in Table 9. Application of the random forest model to this cohort showed good predictive performance with an integrated Brier Score of 0.202.

When the inventors analysed all patients from KCH together without differentiating whether they had history of OHE, the C-index of the AMMON-OHE model was 0.803 (se 0.024), while C-index of MELD and CP were 0.683 (se 0.033) and 0.721 (se 0.026). Application of the AMMON-OHE random forest model showed an integrated Brier Score of 0.161.

### Discussion

This study successfully developed and validated the AMMON-OHE model using ammonia levels and other readily available clinical and biochemical variables to stratify the risk for development of an episode (or first episode) of OHE. Although abnormal PHES test results were associated with OHE and mortality in univariable analyses, abnormal CFF was not, and neither were independent predictors of OHE on multivariable analyses. The addition of high AMM-ULN to the PHES test significantly improved its ability to stratify OHE and mortality risk. However, the AMMON-OHE model that included sex, diabetes, albumin, creatinine and AMM-ULN was found to perform better at prediction of an episode (or first episode) of OHE and mortality than the traditional neuropsychological or psychophysical tests, PHES and CFF, even with the addition of AMM-ULN for the prediction of an episode (or first episode) of OHE and mortality.

OHE is one of the major causes of hospital admissions in patients with decompensated cirrhosis, which impacts significantly on healthcare costs and quality of life of afflicted patients and is a massive burden on caregivers [25]. Beyond the morbidity that OHE induces, its occurrence carries a poor prognosis, with mortality estimated to be 40% at 1-year in the literature [26] and, accordingly, 41% in the training data used by the inventors. As OHE is potentially preventable [29], it is important to detect candidates who would benefit from treatment. The inventors found that patients with abnormal PHES are more likely to develop OHE. However, CFF performance did not correlate with the development of OHE. In addition, although in the univariable analyses, PHES was a significant predictor of both OHE and mortality, abnormality of PHES alone was not an independent predictor of either OHE or mortality in the multivariable analyses. Applying the most accepted cut-off values of <−4 for PHES score and <39 Hz for CFF, a total of 41% of patients showed abnormal PHES while only 34% of patients had altered CFF. In addition, only around 50% of the patients that presented an abnormal test also showed an abnormal result in the other test, confirming low concordance, as has been reported previously (18% to 86%) [28]. These data reflect the discordant and widely variable results described in a recent systematic review, which found that abnormal PHES was associated with OHE only on univariate analysis in 6 studies, with low sensitivities and specificities of 41-77% and 65-75%, respectively. For CFF, several studies have explored its ability to predict OHE with large discrepancies in results [15].

Moreover, most clinicians do not routinely use neuropsychological or psychophysical tests in cirrhosis patients because performance of the test is time-consuming, requires training, are not reimbursed and the tools are not widely available [15]. The AMMON-OHE machine model developed by the inventors uses variables which are easily obtainable from, for example, questionnaires and blood tests. The machine model may assess the probability of a subject developing OHE based on input variables and therefore provides a more efficient method for predicting the risk of developing OHE in cirrhosis patients.

As ammonia has recently been shown to be an independent factor predicting the risk of development of OH E [13], the inventors investigated whether it could improve the performance of the PHES and CFF tests. The results of this study confirmed that elevated ammonia was an independent predictor for the development of OHE and mortality. Indeed, the data showed that patients with hyperammonaemia have a 2-4 times higher risk than patients with normal ammonia levels not only for developing an episode (or first episode) of OHE but also for mortality, see Table 6. To better understand the relative contribution of abnormalities of neuropsychological or psychophysical tests and ammonia levels in defining risk of future OHE and mortality, the inventors created 4 groups according to test performance and ammonia levels. The predictor with highest accuracy for the occurrence of OH E was the addition of high ammonia levels to an abnormal PHES, see FIG. 5.

As neither of the PHES or CFF tests evaluated in this study showed a good predictive ability for the development of OHE, the inventors evaluated whether other clinical or biochemical variables were important in doing so. Serum albumin level, creatinine, presence of diabetes and sex along with ammonia were associated independently with the risk of developing OHE. This observation provided the opportunity to develop a new prognostic model, the AMMON-OHE model, which, for example, could be used at the bed side in routine clinical practice for the detection of patients at high risk of OHE. The components of the AMMON-OHE model are all biologically implicated in the pathogenesis of OHE. Ammonia has been shown in several studies to be central in the pathogenesis of OHE and an increase in its concentration is required for the diagnosis of OHE [29]. A recent systematic review and meta-analysis showed that albumin administration improves OHE and reduces mortality in patients with cirrhosis and OHE [30]. Ammonia is generated and utilized in several organs including not only the brain but also the kidneys and the muscles, which can explain why patients with impaired kidney function and lower muscle mass have a higher risk of OHE. Moreover, comorbidities such as diabetes have been widely implicated in the development of brain dysfunction [31-32].

The AMMON-OHE model was tested in two independent cohorts of outpatients with cirrhosis showing both a high C-index (better than the traditional CP and MELD scores), and a low integrated Brier Score for development of OHE. Application of the AMMON-OHE random forest model from the derivation sets to the validation cohorts further demonstrated the optimal performance of the model and the central importance of AMM-ULN as a prediction variable. Moreover, further analyses of patients with previous OHE showed that the AMMON-OHE was also useful for the prediction of recurrent OHE. The availability of an online App may allow clinicians to assess the risk of development of OHE rapidly in a clinical setting. As there are no drugs shown to prevent an episode (or first episode) of OHE, the AMMON-OHE model may be useful for the selection of patients for clinical trials or as a companion biomarker for the better use of currently available drugs and development of future therapies and/or novel drugs.

The results derived from the available data should be interpreted considering its strengths and limitations. The patient cohorts were derived from three independent centres and patients presented different baseline characteristics, which is a potential limitation. However, the findings are based on 3 well-characterized patient cohorts with prospectively evaluated outcomes and frailty statistical methods were used to minimize cohort-related bias. Moreover, the results were externally validated in two other independent liver units with different baseline characteristics showing that the model can be applied to patients with different disease severity.

The inventors surprisingly found that the results of their study do not support the routine use of PHES and CFF tests to define the risk of development of OHE. Instead, the AMMON-OHE model, which uses readily available clinical and biochemical parameters including sex, diabetes, albumin, creatinine and AMM-ULN performed significantly better than these traditionally used neuropsychological or psychophysical tests and disease severity scores for the prediction of an episode (or first episode) of OHE and its recurrence. The ability of the AMMON-OHE model in predicting an episode (the first episode) of OHE was validated in two independent cohorts and hence, may be used to identify stable, cirrhotic patients at risk of developing OHE. This novel model may be readily adopted in clinical practice as a substitute for neuropsychological or psychophysical tests, to identify patients with cirrhosis at high risk of developing OHE and can also be used for patient selection for clinical trials of existing or novel drugs.

The AMMON-OHE machine learning model may further be used for subjects with acutely decompensated cirrhosis, and changes in the repeated measurements of a subject using the model may be used to evaluate risk of OHE, other liver-related complications and mortality.

Overt hepatic encephalopathy (OHE) occurs in about 30% of cirrhosis patients and its occurrence is associated with considerable morbidity and high risk of mortality. Current models in the prior art define the risk of OHE based on neuropsychometric tests, which require clinical expertise, time and have relatively low sensitivity and specificity. The inventors have demonstrated that hyperammonemia is an independent predictor of hospitalization with liver-related complications and mortality and developed and validated the AMMON-OHE model using machine learning approach, which included ammonia and other readily available clinical variables. The inventors have shown that the machine model outperforms neuropsychometric tests.

The inventors found that a change in the output of the AMMON-OHE model, that is for example the risk of developing OHE over time, may be associated with either increased or reduced risk of an episode (or first episode) of OHE and it may be used to define susceptible patients that may benefit from preventative therapies. For example, the model may be used to determine whether reduction in the output of the model, that is the risk of a subject developing OHE, at 3-months is associated with lower risk of development of OHE in outpatients with cirrhosis.

The changes in the output of the AMMON-OHE model may also be used to predict the risk of recurrent OHE, other liver-related complications and mortality and its impact on quality of life. For example, there may be a relationship between the AMMON-OHE model and other pathophysiological factors associated with liver-related complications.

Venous ammonia levels and markers of liver and kidney function may be measured to calculate expected median survival time free of OHE according to the AMMON-OHE model at baseline, 3 and 6 months of follow-up. Nutritional parameters, quality of life questionnaires and markers of systemic inflammation may be obtained at the same time points.

To further validate the AMMON-OHE model, test patients may be prospectively followed up to 1 year to register hospitalizations with liver-related complications and mortality. Prediction performance of the AMMON-OHE model may be evaluated using area under Receiver Operating characteristic.

### Hardware Description

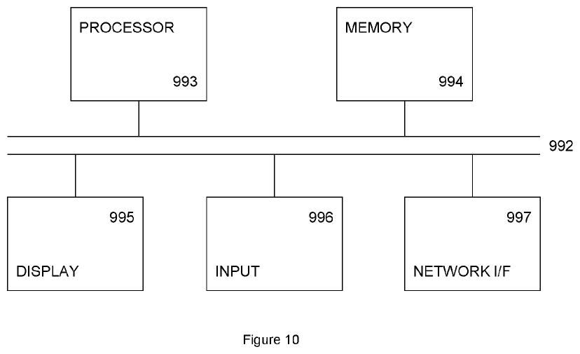

FIG. 10 is a block diagram of a computing device, such as medical test equipment or data storage server, which embodies the present invention, and which may be used to implement a method of an embodiment of a machine learning model assessing the probability of a subject developing OHE or a training method, both as described herein. The computing device comprises a processor 993, and memory, 994. Optionally, the computing device also includes a network interface 997 for communication with other computing devices, for example with other computing devices of invention embodiments.

For example, an embodiment may be composed of a network of such computing devices. Optionally, the computing device also includes one or more input mechanisms such as keyboard and mouse 996, and a display unit such as one or more monitors 995. The components are connectable to one another via a bus 992.

The memory 994 may include a computer readable medium, which term may refer to a single medium or multiple media (e.g., a centralized or distributed database and/or associated caches and servers) configured to carry computer-executable instructions or have data structures stored thereon. Computer-executable instructions may include, for example, instructions and data accessible by and causing a general purpose computer, special purpose computer, or special purpose processing device (e.g., one or more processors) to perform one or more functions or operations. Thus, the term “computer-readable storage medium” may also include any medium that is capable of storing, encoding or carrying a set of instructions for execution by the machine and that cause the machine to perform any one or more of the methods of the present disclosure. The term “computer-readable storage medium” may accordingly be taken to include, but not be limited to, solid-state memories, optical media and magnetic media. By way of example, and not limitation, such computer-readable media may include non-transitory computer-readable storage media, including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices).

The processor 993 is configured to control the computing device and execute processing operations, for example executing code stored in the memory to implement the functions of training or querying the machine learning mode, for example executing R functions, as 1 described here and in the claims. The memory 994 stores data (such as a Random Survival Forest, RSF, model or R project) being read and written by the processor 993. As referred to herein, a processor may include one or more general-purpose processing devices such as a microprocessor, central processing unit, or the like. The processor may include a complex instruction set computing (CISC) microprocessor, reduced instruction set computing (RISC) microprocessor, very long instruction word (VLIW) microprocessor, or a processor implementing other instruction sets or processors implementing a combination of instruction sets. The processor may also include one or more special-purpose processing devices such as an application specific integrated circuit (ASIC), a field programmable gate array (FPGA), a digital signal processor (DSP), network processor, or the like. In one or more embodiments, a processor is configured to execute instructions for performing the operations and steps discussed herein.

The display unit 995 may display a representation of data stored by the computing device and may also display a cursor and dialog boxes and screens enabling interaction between a user and the programs and data stored on the computing device. The input mechanisms 996 may enable a user to input data and instructions to the computing device, or the display unit may incorporate a touchscreen for input of the variables. For example, the display unit may display an application which may take as an input: AMM-ULN and Albumin (g/dL) and optionally Sex, Diabetes Miletus status and Creatinine (mg/dL) of a subject and output on a screen a graph and/or table with a probability or score of developing OHE.

The network interface (network I/F) 997 may be connected to a network, such as the Internet, and is connectable to other such computing devices via the network. The network I/F 997 may control data input/output from/to other apparatus via the network. Other peripheral devices such as microphone, speakers, printer, power supply unit, fan, case, scanner, trackerball etc may be included in the computing device.

Methods embodying the present invention may be carried out on a computing device such as that illustrated in FIG. 10. Such a computing device need not have every component illustrated in FIG. 10, and may be composed of a subset of those components. A method embodying the present invention may be carried out by a single computing device in communication with one or more data storage servers via a network. The computing device may be a data storage itself storing the machine learning model. For example, the computing device may store survival functions for each node of each tree in a random survival forest or may store the ensemble survival function.

A method embodying the present invention may be carried out by a plurality of computing devices operating in cooperation with one another. One or more of the plurality of computing devices may be a data storage server storing at least a portion of the machine learning model, as above. Another device may be a portable test device.

### Portable Test Device

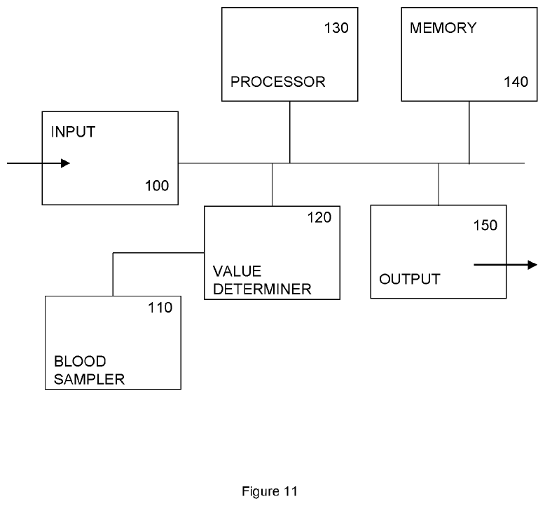

One of the uses of the model is as a companion diagnostic to help decide in taking ammonia-lowering therapies for the treatment of HE, as described above. For these purposes, a portable test device may be provided to measure the therapeutic effect. The device may be hand held. Such a portable test device is shown schematically in FIG. 11. The testing device includes an input part 100 such as a touch screen or keyboard, for the clinician or other user to input the patient data in terms of input values of gender and diabetes status, and also a patient identifier, and any other salient information.

A blood sampler 110 is used to take a blood sample, and a value determiner 120 derives the values of blood ammonia level, blood albumin level, and blood creatinine level for the subject. Construction of such testing stations is known in the art.

For example, the blood test sampler may be a strip or disc analyser and/or a blood container with a value determiner configured to detect a specified variable. For example, the value determiner may be a detector configured to detect ammonia and/or albumin and/or creatinine levels.

The value determiner takes or accepts a subject's blood, from the sampler and determines values for blood ammonia level, blood albumin level, and optionally blood creatinine level for the subject. The value determiner may function using chemical detection, immunoassay, bioassay and/or spectrophotometry.

Processor 130 and memory 140 use all the input values in a previously generated RSF (or other mathematical model) as described herein to calculate the risk. An output 150 such as a screen, for example the touch screen if there is one, may be used to output the risk of OHE, or the risk may be sent across a network (for example to a server or PC to store and display the input data and results) via a network interface.

### REFERENCES

1. Arguedas M R, DeLawrence T G, McGuire B M. Influence of hepatic encephalopathy on health-related quality of life in patients with cirrhosis. Dig Dis Sci. 2003 August; 48(8):1622-6.
2. Tapper E B, Halbert B, Mellinger J. Rates of and Reasons for Hospital Readmissions in Patients With Cirrhosis: A Multistate Population-based Cohort Study. Clin Gastroenterol Hepatol. 2016 August; 14(8):1181-1188.e2.
3. Romero-Gómez M, Boza F, Garcia-Valdecasas M S, Garcia E, Aguilar-Reina J. Subclinical hepatic encephalopathy predicts the development of overt hepatic encephalopathy. Am J Gastroenterol. 2001 September; 96(9):2718-23.
4. Tapper E B. Predicting Overt Hepatic Encephalopathy for the Population With Cirrhosis. Hepatology. 2019 July; 70(1):403-409.
5. Weissenborn K. Diagnosis of minimal hepatic encephalopathy. J Clin Exp Hepatol. 2015 March; 5(Suppl 1):S54-9.
6. Vilstrup H, Amodio P, Bajaj J, Cordoba J, Ferenci P, Mullen K D, et al. Hepatic encephalopathy in chronic liver disease: 2014 Practice Guideline by the American Association for the Study of Liver Diseases and the European Association for the Study of the Liver. Hepatology. 2014 August; 60(2):715-35.
7. European Association for the Study of the Liver. EASL Clinical Practice Guidelines on the management of hepatic encephalopathy. J Hepatol. 2022 Jun. 7:S0168-8278(22)00346-4.
8. Sabbah-Talasazan L, Piryatinsky I. Neuropsychological impairment in Hashimoto's encephalopathy: A case report and literature review. Appl Neuropsychol Adult. 2018 November-December; 25(6):572-580.
9. Pitel A L, Zahr N M, Jackson K, et al. Signs of preclinical Wernicke's encephalopathy and thiamine levels as predictors of neuropsychological deficits in alcoholism without Korsakoff's syndrome. Neuropsychopharmacology. 2011 February; 36(3):580-8.
10. Bosoi C R, Rose C F. Identifying the direct effects of ammonia on the brain. Metab Brain Dis. 2009 March; 24(1):95-102.
11. Angelova P R, Kerbert A J C, Habtesion A, et al. Hyperammonaemia induces mitochondrial dysfunction and neuronal cell death. JHEP Rep. 2022 May 23; 4(8):100510.
12. Balzano T, Dadsetan S, Forteza J, et al. Chronic hyperammonemia induces peripheral inflammation that leads to cognitive impairment in rats: Reversed by anti-TNF-α treatment. J Hepatol. 2020 September; 73(3):582-592.
13. Tranah T H, Ballester M P, Carbonell-Asins J A, et al. Plasma ammonia levels predict hospitalisation with liver-related complications and mortality in clinically stable outpatients with cirrhosis. J Hepatol. 2022 Jul. 22:S0168-8278(22)02947-6.
14. Ferenci P, Lockwood A, Mullen K, Tarter R, Weissenborn K, Blei A T. Hepatic encephalopathy—definition, nomenclature, diagnosis, and quantification: final report of the working party at the 11th World Congresses of Gastroenterology, Vienna, 1998. Hepatology. 2002 March; 35(3):716-21.
15. Hansen M K G, Kjorgaard K, Eriksen L L, Grønkjor LL, Mikkelsen A C D, Sandahl T D, et al. Psychometric methods for diagnosing and monitoring minimal hepatic encephalopathy—current validation level and practical use. Metab Brain Dis. 2022 March; 37(3):589-605.
16. Kamath P S, Wiesner R H, Malinchoc M, et al. A model to predict survival in patients with end-stage liver disease. Hepatology 2001; 33:464-470.
17. Pugh R N, Murray-Lyon I M, Dawson J L, et al. Transection of the oesophagus for bleeding oesophageal varices. Br J Surg 1973; 60:646-649.
18. Therneau T. A Package for Survival Analysis in R. R package version 3.4-0, 2022. https://CRAN.R-project.org/package=survival
19. Ishwaran H K U. Fast Unified Random Forests for Survival, Regression, and Classification (RF-SRC). R package version 3.0.0. https://cran.r-project.org/package=randomForestSRC.2022
20. Mogensen U B, Ishwaran H, Gerds T A. Evaluating Random Forests for Survival Analysis using Prediction Error Curves. J Stat Softw 2012; 50(11): 1-23.
21. Nabi E, Bajaj J S. Useful tests for hepatic encephalopathy in clinical practice. Curr Gastroenterol Rep. 2014 January; 16(1):362.
22. Tranah T H, Ballester M P, Carbonell-Asins J A, et al. Plasma ammonia levels predict hospitalisation with liver-related complications and mortality in clinically stable outpatients with cirrhosis. J Hepatol. 2022 Jul. 22:S0168-8278(22)02947-6.
23 Vilstrup H, Amodio P, Bajaj J, Cordoba J, Ferenci P, Mullen K D, et al. Hepatic encephalopathy in chronic liver disease: 2014 Practice Guideline by the American Association for the Study of Liver Diseases and the European Association for the Study of the Liver. Hepatology. 2014 August; 60(2):715-35.
24. Yoav B, Yosef H. Controlling the False Discovery Rate: A Practical and Powerful Approach to Multiple Testing. J R Stat Soc: Series B (Methodological), 1995; 57(1), 289-300.
25. Bajaj J S, Wade J B, Gibson D P, Heuman D M, Thacker L R, Sterling R K, et al. The multi-dimensional burden of cirrhosis and hepatic encephalopathy on patients and caregivers. Am J Gastroenterol. 2011 September; 106(9):1646-53.
26. Stewart C A, Malinchoc M, Kim W R, et al. Hepatic encephalopathy as a predictor of survival in patients with end-stage liver disease. Liver Transpl. 2007 October; 13(10):1366-71.
27. Sharma P, Sharma B C, Agrawal A, Kamath P S. Primary prophylaxis of overt hepatic encephalopathy in patients with cirrhosis: an open labeled randomized controlled trial of lactulose versus no lactulose. J Gastroenterol Hepatol. 2012 August; 27(8):1329-35.
28. Goldbecker A, Weissenborn K, Hamidi Shahrezaei G, Afshar K, Rümke S, Barg-Hock H, et al. Comparison of the most favoured methods for the diagnosis of hepatic encephalopathy in liver transplantation candidates. Gut. 2013 October; 62(10):1497-504.
29. Jalan R, Rose C F. Heretical thoughts into Hepatic Encephalopathy. J Hepatol. 2022 Mar. 28:S0168-8278(22)00183-0.
30. Is B, Bombassaro I Z, Tovo C V, de Mattos A Z, Ahlert M, Chiesa T, et al. Albumin in the management of hepatic encephalopathy: A systematic review and meta-analysis. Ann Hepatol. 2021 December; 26:100541.
31. Kjmrgaard K, Mikkelsen A C D, Wernberg C W, Grønkjor LL, Eriksen P L, Damholdt M F, et al. Cognitive Dysfunction in Non-Alcoholic Fatty Liver Disease-Current Knowledge, Mechanisms and Perspectives. J Clin Med. 2021 Feb. 9; 10(4):673.
32. Ballester M P, Gallego J J, Fiorillo A, Casanova-Ferrer F, Giménez-Garzó C, Escudero-Garcia D, et al. Metabolic syndrome is associated with poor response to rifaximin in minimal hepatic encephalopathy. Sci Rep. 2022 Feb. 14; 12(1):2463.

1. A computer implemented method of determining risk of a subject developing overt hepatic encephalopathy, OHE, over time including:
accepting input of values for blood ammonia level and blood albumin level and optionally the gender of the subject, diabetes status of the subject, and blood creatinine level for the subject into a mathematical model;
the mathematical model assessing and outputting the risk of the subject developing OHE over time.

2. A method according to claim 1, further comprising a step of outputting the risk as a survival function, for example using a graph of probability of developing OHE, and/or mortality, against time, for instance in a Kaplan Meier curve and/or a table of probability of developing OHE, and/or mortality, against time.

3. A method according to claim 1 wherein the model is a Machine Learning, ML, model such as a Random Survival Forest, RSF, model preferably developed using sampling, such as bootstrapping and/or bagging techniques.

4. A method according to claim 3, wherein assessing the probability of the subject developing OHE over time uses an ensemble survival function generated from subject terminal nodes of trees in the RSF which are associated with the subject, the subject terminal nodes corresponding to the subject inputs.

5. A method according to claim 1 wherein the blood ammonia level is input as a ratio of a blood ammonia level of the subject to an upper limit of normal blood ammonia level, AMM-ULN.

6. A method according to claim 1 wherein the subject has cirrhosis and a previous episode of OHE and the method assesses the risk of recurrent OHE; or wherein the subject has stable cirrhosis and the model predicts a first episode of OHE.

7. A method according to claim 1, wherein the model produces a score, for example based on the probability of developing OHE within the first year of testing.

8. A method according to claim 1, further comprising use of the mathematical model to identify patients requiring specific therapy to prevent the occurrence of OHE, preferably by comparing the risk of the subject developing OHE over time with a threshold and outputting a recommendation or risk categorisation.

9. A method according to claim 1, further comprising use of the mathematical model as a surrogate marker of response to and/or failure of treatment, preferably by carrying out the method for a subject at predetermined time intervals and comparing the risk of the subject developing OHE over time.

10. A method according to claim 1, wherein the input uses a web-based interface or a desktop or a portable test device application.

11. A portable test device to determine risk of a subject developing overt hepatic encephalopathy, OHE, over time comprising:
a blood test sampler and value determiner to take a subject's blood and determine values for blood ammonia level, blood albumin level, and optionally blood creatinine level for the subject;
optionally an input part such as a touch screen or keyboard for optionally inputting the gender of the subject and a diabetes status of the subject;
a processor or link to a processor to accept the values for blood ammonia level and blood albumin level, and optionally the gender of the subject, diabetes status of the subject and blood creatinine level and process them in a mathematical Model which assesses the probability of the subject developing OHE based on the time to OHE;
an output part, such as a screen or network interface, to output the risk.

12. A portable test device according to claim 11, wherein the output part outputs the risk as a survival function, for example as a displayed graph and/or a table of probability of OHE against time, for example based on the probability of developing OHE within the first year of testing.

13. A computer implemented method of training a mathematical model to determine risk of a subject developing overt hepatic encephalopathy, OHE, over time including:
inputting, for each of a cohort of subjects, values for blood ammonia level and blood albumin level and optionally the gender of the subject, diabetes status of the subject and, and blood creatinine level for the subject into training software to train a mathematical Model;
inputting, for each of the cohort of subjects, an indication if the subject did or did not develop OHE, and an associated time;
creating the mathematical model using the input values.

14. A method according to claim 13, wherein the model is a Machine Learning, ML, model such as a Random Survival Forest, RSF, model preferably developed using sampling, such as bootstrapping and/or bagging techniques.

15. A computer program, comprising instructions which when the program is executed on a portable test device or processing device, cause the portable test device or processing device to carry out the method of claim 1.

16. A method according to claim 2 wherein the model is a Machine Learning, ML, model such as a Random Survival Forest, RSF, model preferably developed using sampling, such as bootstrapping and/or bagging techniques.

17. A computer program, comprising instructions which when the program is executed on a portable test device or processing device, cause the portable test device or processing device to carry out the method claim 13.
